(19) United States
(12) Patent Application Publication
(10) Pub. No.: US 2004/0073303 A1
(43) Pub. Date: Apr. 15, 2004

(54) RADIAL INTRASTROMAL CORNEAL INSERT AND A METHOD OF INSERTION

(75) Inventors: DAVID J. SCHANZLIN, LA JOLLA, CA (US); STEVEN M. VERITY, ST LOUIS, MO (US); THOMAS A. SILVESTRINI, ALAMO, CA (US); ROBERT A. PROUDFOOT, SANTA CLARA, CA (US)

Correspondence Address: Antoinette F. Konski, McCutchen Doyle Brown & Enersen LLP, Three Embaracadero Center, Suite 1800, San Francisco, CA, 94111-4067, US

(73) Assignee: HARRY J. MACEY

(21) Appl. No.: 08/993,696

(22) Filed: Dec. 18, 1997

Related U.S. Application Data
(63) Continuation-in-part of application No. 08/662,781, filed on Jun. 7, 1996, now abandoned.
Application No. 08/662,781 is a continuation-in-part of application No. 08/485,400, filed on Jun. 7, 1995, now abandoned.

(51) Int. Cl. A61F 2/14

(52) U.S. Cl. 623/5.16; 623/5.11; 606/107; 606/166

(57) ABSTRACT

The subject invention relates to an intrastromal corneal insert designed to be meridionally situated in an interlamellar pocket or channel made within the cornea of a mammalian eye. The insert has a shape which, when inserted into the cornea, has a significant meridional dimension and may be used to adjust corneal curvature and thereby correct or improve vision abnormalities such as hyperopia. The inserts may also have a circumferential component to their configuration to allow concurrent correction of other vision abnormalities. The radial insert may be made of a physiologically compatible material, e.g., one or more synthetic or natural, soft, firm, or gelatinous polymers. In addition, the insert or segment may be used to deliver therapeutic or diagnostic agents to the corneal interior or to the interior of the eye.

One or more of the radial inserts of this invention typically are inserted into the cornea so that each subtends a portion of the meridian of the cornea outside of the cornea's central area, e.g., the area through which vision is achieved, but within the cornea's frontal diameter. Typically, the insert is used in arrays of two or more to correct specific visual abnormalities, but may be used in isolation when such is called for. The invention also includes both a minimally invasive procedure for inserting one or more of the devices into the cornea using procedures beginning within the cornea as well as procedures beginning in the sclera.

### CROSS-REFERENCE TO RELATED APPLICATIONS

This application is a continuation-in-part of co-pending application Ser. No. 08/662,781, filed Jun. 7, 1996, which is a continuation-in-part application of co-pending U.S. application Ser. No. 08/485,400, Filed Jun. 7, 1995.

### FIELD OF THE INVENTION

This invention is an intrastromal corneal insert designed to be placed into an interlamellar pocket or channel made within the cornea of a mammalian eye. The insert has a shape which, when inserted into the cornea, has a significant radial or meridional dimension and may be used to adjust corneal curvature and thereby correct or improve vision abnormalities such as hyperopia. The inserts may also have a circumferential component to their configuration to allow concurrent correction of other vision abnormalities. The radial insert may be made of a physiologically compatible material, e.g., one or more synthetic or natural, soft, firm, or gelatinous polymers. In addition, the insert or segment may be used to deliver therapeutic or diagnostic agents to the corneal interior or to the interior of the eye.

One or more of the radial inserts of this invention typically are inserted into the cornea so that each subtends a portion of the meridian of the cornea outside of the cornea's central area, e.g., the area through which vision is achieved, but within the cornea's frontal diameter. Typically, the insert is used in arrays of two or more to correct specific visual abnormalities, but may be used in isolation when such is called for. The invention also includes both a minimally invasive procedure for inserting one or more of the devices into the cornea using procedures beginning within the cornea as well as procedures beginning in the sclera. The thus-corrected eye itself forms another aspect of the invention.

### BACKGROUND OF THE INVENTION

Anomalies in the overall shape of the eye can cause visual disorders. Hyperopia (“farsightedness”) occurs when the front-to-back distance in the eyeball is too short. In such a case, parallel rays originating greater than 20 feet from the eye focus behind the retina. Although minor amounts of hyperopia can be resolved in the human eye by a muscular action known as “accommodation”, aging often compromises the ability of the eye adequately to accommodate. In contrast, when the front-to-back distance of eyeball is too long, myopia (“nearsightedness”) occurs and the focus of parallel rays entering the eye occurs in front of the retina. Astigmatism is a condition which occurs when the parallel rays of light do not focus to a single point within the eye, but rather have a variable focus due to the fact that the cornea refracts light in a different meridian at different distances. Some degree of astigmatism is normal, but where it is pronounced, the astigmatism must be corrected.

Hyperopia, myopia, and astigmatism are usually corrected by glasses or contact lenses. Surgical methods for the correction of such disorders are known. Such methods include radial keratotomy (see, e.g., U.S. Pat. Nos. 4,815,463 and 4,688,570) and laser corneal ablation (see, e.g., U.S. Pat. No. 4,941,093).

Another method for correcting those disorders is through the implantation of polymeric rings (intrastromal corneal rings) in the eye's corneal stroma to change the curvature of the cornea. Previous work involving the implantation of polymethylmethacrylate (PMMA) rings, allograft corneal tissue, and hydrogels is well documented. One of the ring devices involves a split ring design which is inserted into a channel previously dissected in the stromal layer of the cornea. A minimally invasive incision is used both for producing the channel and for inserting the implant. See, for instance, the use of PMMA intrastromal rings in U.S. Pat. No. 4,452,235 to Reynolds; U.S. Pat. No. 4,671,276 to Reynolds; U.S. Pat. No. 4,766,895 to Reynolds; and U.S. Pat. No. 4,961,744 to Kilmer et al. These documents suggest only the use of intrastromal corneal rings which completely encircle the cornea.

The use of soft polymers as intrastromal inserts is not widely known. For instance, U.S. Pat. Nos. 5,090,955 and 5,372,580, to Simon, suggest an intrastromal corneal ring which is made by introducing a settable polymer or gel into an intrastromal channel which has been previously made and allowing the polymer to set. This procedure does not allow the surgeon to specify the precise size of the resulting ring nor is it a process which allows precise control of the pathway of the flowing polymer within the eye since the gel must simply conform to the shape of the intrastromal channel. However, it does show the concept of using arcuate channels containing a gel-based insert centered on the cornea.

Temirov et al., “Refractive circular tunnel keroplasty in the correction of high myopia”, Vestnik Oftalmologii 1991: 3-21-31, suggests the use of collagen thread as intrastromal corneal ring material.

These publications do not suggest the introduction of polymeric inserts having significant radial or meridional dimensions into the cornea for the correction of various visual aberrations. The publications do not imply that the devices may be used to introduce therapeutic or diagnostic materials into the corneal intrastromal space.

### SUMMARY OF THE INVENTION

This invention is a polymeric insert suitable for insertion between the lamella of the corneal stroma. The insert may be of any of a variety of shapes, including straight, lozenge-shaped, arcuate, cross-shaped, anchor-shaped, button-shaped or but in any event has a significant radial or meridional component when inserted into the cornea. The insert may be used in isolation, in arrays of isolated multiple inserts, in cooperative multiples, as segments in a larger assemblage encircling at least a portion of the cornea, or as assemblages to form constructs of varying thickness.

This invention is a method of inserting a polymeric insert into a cavity formed between the lamella of the corneal stroma. The insert may be of one or more synthetic or natural polymers, hydrophilic or hydrophobic, or may be a hybrid device comprising layered materials. Optionally, the insert may contain filamentary material in the form of a single or multiple threads, random-included filaments, or woven mattes to reinforce the insert during, e.g., insertion or removal from the intrastromal channel.

The insert may be hollow and may be filled with a biologic agent, drug or other liquid, emulsified, or time-release eye treatment or diagnostic material. The insert may contain a gel, viscous, or visco-elastic material which remains in such a state after introduction. The insert may be a gel. The insert may be an injectable solid which deforms upon introduction but conforms to the form of the previously formed injection site in the cornea upon relaxation at the chosen site.

When a hybrid, the inner portion may comprise variously a composite of low modulus polymers or a single low modulus polymer. The inner portion may also comprise a polymeric material which is polymerized in situ after introduction into the hollow center layer.

These inventive segmented inserts may be introduced into the corneal stroma using techniques involving the steps of providing an intrastromal pocket or channel. The intrastromal pocket into which the insert is placed is, in its most simple variation, a pocket having an opening somewhere in its length into which the insert is placed. The pocket typically will have its outer end near the outer periphery of the cornea and proceeds from there towards the center of the cornea but stopping short of the sight area of the cornea. If the insert has a circumferential component as well, the pocket may be modified to include a channel which traverses at least a portion of the circumcorneal rotation to accommodate that circumferential dimension.

Specific indications, such as astigmatism, may also be rectified by insertion of one or more of the inserts into a partial intrastromal channel to steepen the center of the corneal surface. The inserts need not be of the same size, thickness, or configuration.

If hydratable polymers are used, they may be hydrated before or after introduction into the intrastromal pockets or channels created by the surgical device used to introduce these devices into the eye. If the outer layer is hydrated before insertion into the eye, the final size of the insert may be set before that insertion. If the hydratable polymers are allowed to hydrate within the corneal space, the device (if appropriate polymers are chosen) will swell within the eye to its final size. If prehydrated, the outer layer often provides a measure of lubricity to the device, allowing it to be inserted with greater ease. Other of the noted low modulus polymers may also provide such lubricity.

### BRIEF DESCRIPTION OF THE DRAWINGS

FIG. 1 is a schematic illustration of a horizontal section of the eye.

FIG. 2 is a schematic illustration of the anterior portion of the eye showing the various layers of the cornea.

FIGS. 3A and 3B show respectively a front view and a cross section of a typical array of intracorneal inserts made according to the invention.

FIG. 3C shows an alternate convention for identifying the relationship between inserts.

FIGS. 4A, 4B, and 4C show typical inserts made according to the invention explaining the conventions and terms used in explaining this invention.

Each of FIGS. 5A and 5B, 6A and 6B, 7A and 7B, and 8A and 8B show respectively a front view (“A” drawing) and a side view (“B” drawing) of various intracorneal inserts made according to the invention. FIGS. 5C and 6C show cross section of the 5B and 6B inserts, respectively.

Each of FIGS. 9 to 15 show perspective views of variations of the inventive insert.

FIGS. 16 and 17 show respectively a front view of two inventive intracorneal inserts placed in circumferential contact with each other.

FIG. 18 shows a front view of a eye having both radial inserts and circumferential segments placed therein.

FIGS. 19A and 19B show respectively a front view and a cross section of a soft, filled intracorneal insert made according to the invention.

FIGS. 20A and 20B show respectively a front view and a cross section of a layered, composite intracorneal insert made according to the invention.

FIGS. 21A to 21D schematically depict a procedure for introducing the inserts into the cornea using a circumferential intrastromal channel.

FIGS. 22A through 22C schematically depict a procedure in which the various radial inserts are introduced using individual meridional incisions.

FIGS. 23 to 24 schematically depict a procedure for introducing the inventive inserts into the cornea through circumferential incisions.

FIGS. 25A-25C schematically depicts an alternative procedure for introducing the inserts.

FIGS. 26A and 26B illustrate a corneal marker.

FIGS. 27A and 27B illustrate an alternative corneal marker.

FIGS. 28A and 28B illustrate another alternative corneal marker.

FIGS. 29A to 29C illustrate a radial pocket-forming instrument.

FIGS. 30A and 30B illustrate a positioning instrument.

FIGS. 31A to 31C schematically depicts a procedure for introducing a gel into meridional pockets in the cornea.

FIGS. 32A and 32B show respectively a side view and a cross section of an insert having a partially tapered end made according to the invention.

FIGS. 33A and 33B show respectively a side view and a cross section of an insert having a fully tapered end made according to the invention.

FIG. 34 shows a front view of a corneal pocketing tool.

FIG. 35 shows a magnified front view of a corneal pocketing tool in operation.

FIG. 36 shows a plan view of a spreader according to the present invention.

FIG. 36A shows a partial view of the spreader of FIG. 36, starting from cut lines A-A.

FIG. 36B is a partial view similar to that of FIG. 8A, but rotated 90 degrees.

FIG. 36C is a sectional view taken along lines C-C in FIG. 5B.

FIG. 36D is a sectional view taken along lines D-D in FIG. 5B.

FIG. 36E is a magnified view of the tip of FIG. 5A starting from cut lines E-E.

FIG. 36F is a modification of the tip shown in FIG. 8A.

FIG. 37 is a front view of a spreader having an alternate hangle orientation.

FIG. 38A is a partial top view showing a spreader tip.

FIG. 38B is a partial top view showing an alternative spreader tip.

### DESCRIPTION OF THE INVENTION

Prior to explaining the details of the inventive devices, a short explanation of the physiology of the eye is needed to appreciate the functional relationship of these intracorneal inserts or segments to the eye.

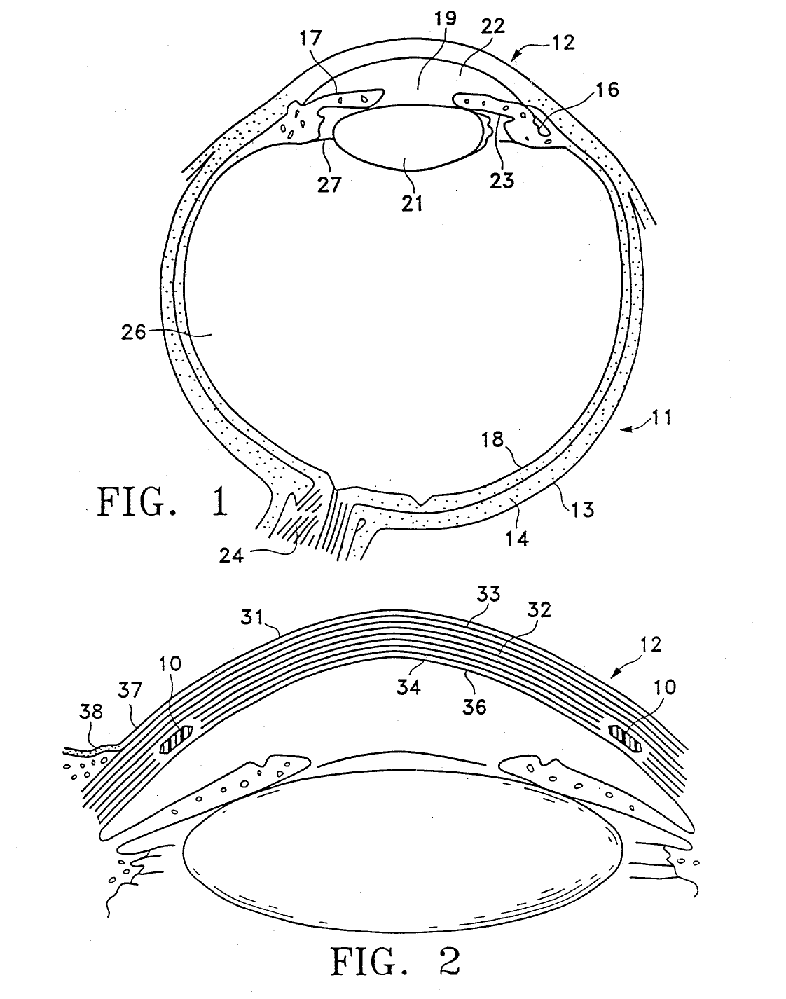

FIG. 1 shows a horizontal cross-section of the eye with the globe (11) of the eye resembling a sphere with an anterior bulged spherical portion representing the cornea (12).

The globe (11) of the eye consists of three concentric coverings enclosing the various transparent media through which the light must pass before reaching the light-sensitive retina (18). The outermost covering is a fibrous protective portion the posterior five-sixths of which is white and opaque and called the sclera (13), and sometimes referred to as the white of the eye where visible to the front. The anterior one-sixth of this outer layer is the transparent cornea (12).

A middle covering is mainly vascular and nutritive in function and is made up of the choroid, ciliary body (16), and iris (17). The choroid generally functions to maintain the retina (18). The ciliary body (16) is involved in suspending the lens (21) and accommodation of the lens. The iris (17) is the most anterior portion of the middle covering of the eye and is arranged in a frontal plane. It is a thin circular disc similar in function to the diaphragm of a camera, and is perforate near its center by a circular aperture called the pupil (19). The size of the pupil varies to regulate the amount of light which reaches the retina (18). It contracts also to accommodation, which serves to sharpen the focus by diminishing spherical aberration. The iris divides the space between the cornea (12) and the lens (21) into an anterior chamber (22) and the posterior chamber (23). The innermost portion of covering is the retina (18), consisting of nerve elements which form the true receptive portion for visual impressions.

The retina (18) is a part of the brain arising as an outgrowth from the fore-brain, with the optic nerve (24) serving as a fiber tract connecting the retina part of the brain with the fore-brain. A layer of rods and cones, lying just beneath a pigmented epithelium on the anterior wall of the retina serve as visual cells or photoreceptors which transform physical energy (light) into nerve impulses.

The vitreous body (26) is a transparent gelatinous mass which fills the posterior four-fifths of the globe (11). At its sides it supports the ciliary body (16) and the retina (18). A frontal saucer-shaped depression houses the lens.

The lens (21) of the eye is a transparent bi-convex body of crystalline appearance placed between the iris (17) and vitreous body (26). Its axial diameter varies markedly with accommodation. A ciliary zonule (27), consisting of transparent fibers passing between the ciliary body (16) and lens (21) serves to hold the lens (21) in position and enables the ciliary muscle to act on it.

Referring again to the cornea (12), this outermost fibrous transparent coating resembles a watch glass. Its curvature is somewhat greater than the rest of the globe and is ideally spherical in nature. However, often it is more curved in one meridian than another giving rise to astigmatism. A central third of the cornea is called the optical zone with a slight flattening taking place outwardly thereof as the cornea thickens towards its periphery. Most of the refraction of the eye takes place through the cornea.

FIG. 2 is a more detailed drawing of the anterior portion of the globe showing the various layers of the cornea (12) making up the epithelium (31). Epithelial cells on the surface thereof function to maintain transparency of the cornea (12). These epithelial cells are rich in glycogen, enzymes and acetylcholine and their activity regulates the corneal corpuscles and controls the transport of water and electrolytes through the lamellae of the stroma (32) of the cornea (12).

An anterior limiting lamella (33), referred to as Bowman's membrane or layer, is positioned between the epithelium (31) and the stroma (32) of the cornea. The corneal stroma (32) are made up of lamellae having bands of fibrils parallel to each other and crossing the whole of the cornea while most of the fibrous bands are parallel to the surface, some are oblique, especially anteriorly. A posterior limiting lamella (34) is referred to as Descemet's membrane. It is a strong membrane sharply defined from the stroma (32) and resistant to pathological processes of the cornea. The endothelium (36) is the most posterior layer of the cornea and consists of a single layer of cells. The limbus (37) is the transition zone between the conjunctiva (38) and sclera on the one hand and the cornea (12) on the other.

With that background in place, our invention centers on the finding that introduction of an insert into the cornea, typically and desirably between the lamellar layers making up the cornea, in a position meridional to the cornea results in an alleviation of hyperopia. Although we do not wish to be bound by theory, we believe that the introduction of these radial segments results in a steepening of the center of the cornea. There may be other beneficial effects to a specific corneal surface, e.g., correction of myopia and astigmatism, but the correction to hyperopia is apparent. The meridional component to the device for correction of hyperopia may also be combined with the effects we have earlier found relating to the introduction of inserts (of this and other designs) circumferentially around the periphery of the cornea to alleviate myopia and similar problems and inserts of varying thickness at the periphery to alleviate astigmatism. The inserts may be teamed with other intrastromal corneal rings or partial rings or segments which are placed circumferentially about the periphery of the cornea so to correct independently a number of different ocular irregularities.

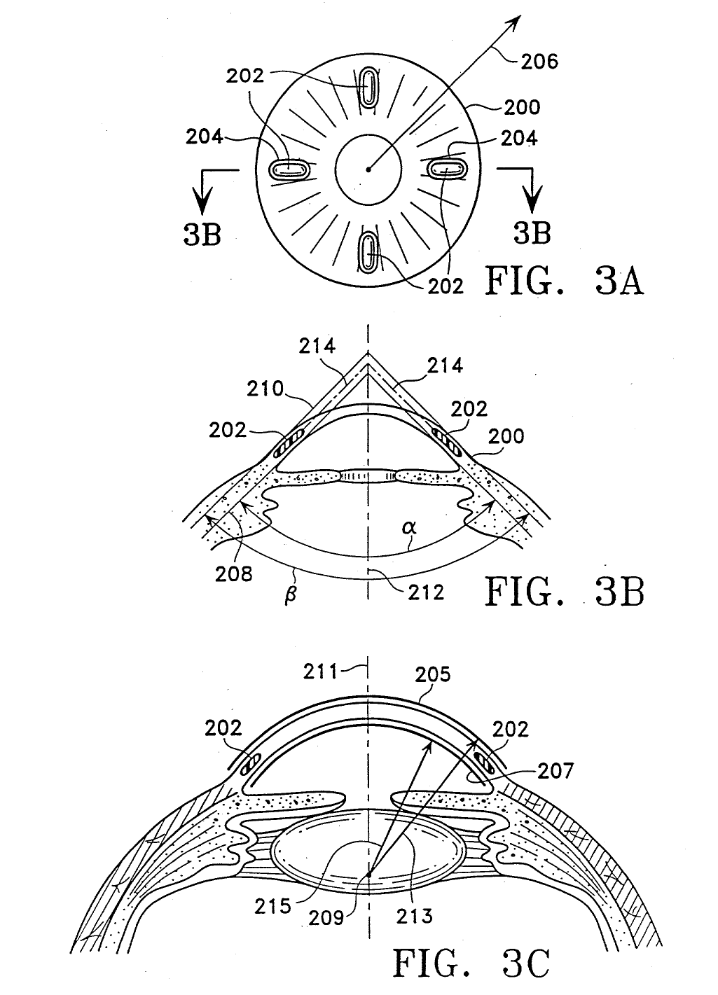

FIG. 3A is a frontal view of the cornea (200) of an eye having four or an “array” of inserts (202) which are located within small meridional pockets (204). The inserts (202) are placed generally on a meridian (206) of the cornea. By “meridian” we mean the typical meaning: the direction of a line beginning at the center of the cornea, as viewed from the front of the eye, and extending outwardly towards the outer circumference of the cornea.

In any case, it is this meridional placement and sizing which is believed to cause the steepening of the center of the cornea (200) as discussed above.

FIG. 3B shows a side view cross section of the cornea (200) of FIG. 3A and has imposed upon it a pair of conic surfaces (208 & 210) sharing a common conic axis (212) and a common conic direction in that the apex of conic surface (208) is within conic surface (210). The volume between the two conic surfaces (208 & 210) forms a mathematical solid in which the inserts (202) lie. The length of the inserts (202), if extended as shown with a dashed line (214), extends toward the common conic axis (212). The cone angles of the two conic surfaces (208 & 210), respectively α and β, typically are independently between 15° and 60°. Although the two conic surfaces are (208 & 210) shown to be approximately parallel, this invention also includes the variation in which the two conic surfaces converge beyond the periphery of the cornea (200).

FIG. 3C shows another explanation of the relationship of the various radial inserts to each other within the cornea. As was the case with the relationship shown in FIG. 3B, one or more inserts (202) may be placed within the cornea as shown frontally in FIG. 3A. FIG. 3C shows the two inserts (202) visible in this side view. Other inserts may also be present in the cornea which are not visible in this view. The inserts are situated with respect to each other in the following manner: two or more such inserts are found between two partial hemispheres or spherical shells (205 and 207) which generally share a common center (209) or center-line (211). The radius (213) of the larger partial hemisphere (205) results in a surface approximating the anterior surface of the cornea. The radius (215) of the smaller partial hemisphere (207) results in a surface approximating the posterior surface of the cornea. The typical length of the larger radius (213) for most human eyes is between 6.7 mm and 9.0 mm. The typical length of the smaller radius (215) for most human eyes is between 5.5 mm and 7.0 mm. The typical thickness of a human cornea (and hence the difference between the length of larger radius (213) and smaller radius (215)) is between 0.4 mm and 0.8 mm. The inserts (204) are situated in the same generally meridional position as depicted in FIG. 3A—that is to say that the insert (204) desirably lies in a position which, if extended, would intersect the centerline (211).

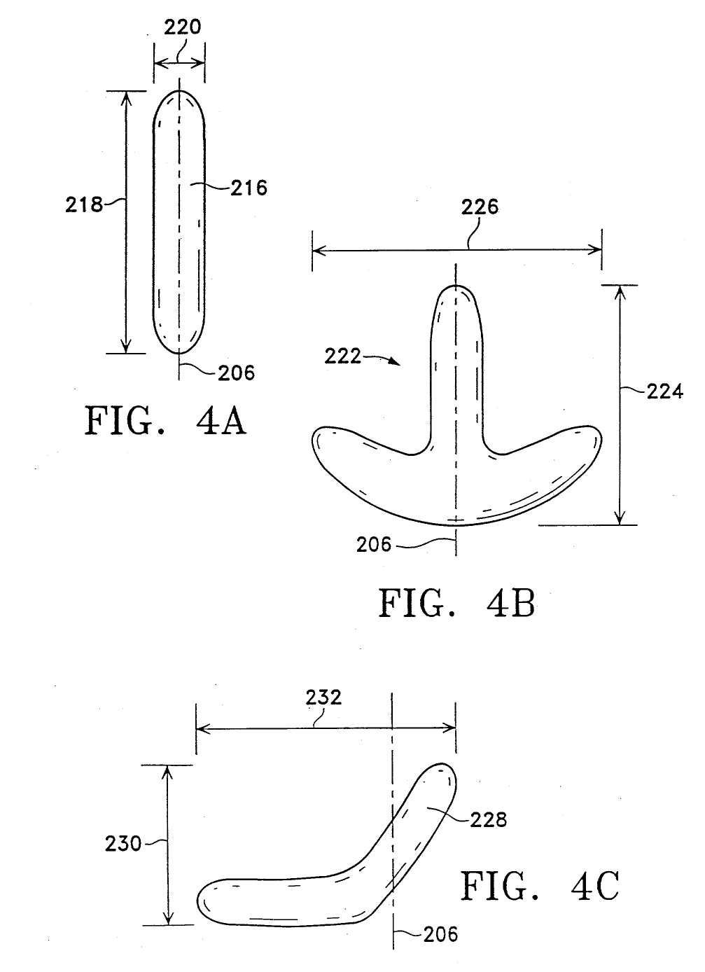

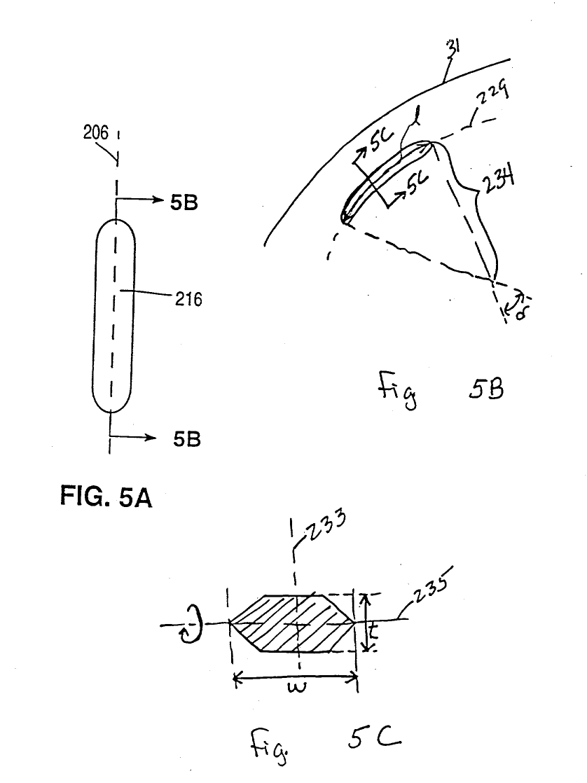

FIGS. 4A, 4B, and 4C show front views of variations of the inventive inserts, provide explanations of the conventions used in defining the meridional and circumferential dimensions, and show general orientation of the inserts in the cornea. For instance, in FIG. 4A is seen one of the simplest forms of inserts made according to the invention. There, the insert (216) has a significant meridional length component (218) lying approximately along corneal meridian (206), and only a small width or circumferential component (220). The ratio of the length of the meridional length component (218) to the width or circumferential component (220) is typically greater than 1.0, preferably 1.5 to 20.0 and, for the simple insert (216) shown in FIG. 4A, may even be 20.0 or more.

FIG. 4B shows an insert (222) having both a significant meridional length component (224) and a significant meridional length component (224) and a significant width or circumferential component (226). The ratio of the length of the meridional length component (224) to the width or circumferential component (226) in the variation shown in FIG. 4B is about 1.0. Its general positioning to the corneal meridian (206) is also depicted in the Figure.

FIG. 4C also shows an inert (228) having both a significant meridional length component (230) and a significant width or circumferential component (232). The ratio of the length of the meridional length component (230) to the width or circumferential component (232) in the variation shown in FIG. 4C is also about 1.0.

The concept of measuring the meridional length of the inserts by observing the length of the insert which falls along a meridian (206) of the cornea should be clear from the examples shown in FIGS. 4A, 4B, and 4C.

FIG. 5A shows a front view of an insert (216) made according to the invention. FIG. 5B shows a side view of the FIG. 5A insert in relation to the anterior corneal surface which follows the external epithelium (31). The side view shows a desirable embodiment in which the insert's centroidal axis follows an intracorneal arc (229) in a direction parallel to a corneal meridian, and if not pliable, exhibits a pre-shaped radius of curvature (234) before implantation, as well as after implantation. Such a device is referred to as “radially arcuate.” The radius of curvature (234) approximates, e.g., lies between, the hemispherical radii (213 and 215) shown in FIG. 3C at the depth of implantation in the cornea into which it is placed. This radius of curvature (i.e., the radius of curvature measured along the centroidal axis of the insert, or that portion of the insert intended to extend generally radially within the cornea) is preferably greater than 5 mm, more preferably greater than 5.5 mm, and typically ranges from 6 to 9 mm. In a preferred embodiment, the radius of curvature ranges from 7 to 8 mm.

FIG. 5B also shows that the insert has a centroidal length (“l”) measured along its centroidal axis at a given radius of curvature (234). This centroidal length “l” subtends an arc having an angle “δ”. This value is referred to herein as the “meridional arc angle”. The value of δ is preferably less than or equal to 90°, and more preferably less than or equal to 45°.

The device of FIG. 5B may have a variety of different cross sectional configurations. FIG. 5C, for example, shows a cross sectional view of the FIG. 5B device having a hexagonal cross section. As shown, the hexagonal cross section of the insert gives rise to two axes; a short axis (233) which lies in the same plane as the meridional component and a long axis (235) perpendicular to this plane. The short axis defines the thickness of the insert, “t” whereas the long axis defines the width of the insert, “w”. As depicted in FIGS. 5B and 5C, the radially arcuate insert subtends a meridional arc around an axis which is generally parallel to the long axis. Variations of the insert in which pliable polymers or gels are used need not be pre-formed in this way. The variation of FIGS. 5A, 5B and 5C desirably tapers to a blunt point at one or both of the ends of the device. Such a configuration allows for ease of insertion and reduces trauma to eye tissue.

The concept of measuring the centroidal length of the insert by observing the length of the insert along the centroidal axis of the insert which extends in the direction of a corneal meridian (206), should be clear from the examples shown in FIGS. 5A, 5B, and 5C.

FIG. 6A shows an anchor-shaped variation (222) of the inventive insert having a radial leg (236) and a circumferential portion (238). Such an insert is referred to as a “combination insert.” Although this variation has a significant radial leg (236), the correction of hyperopia may be secondary in importance. The circumferential portion (238) subtends a certain portion of the circumference of the cornea along an arcuate path around (i.e., perpendicular to) the short axis (235), and is thus said to be “circumferentially arcuate”, and may accordingly effect the correction of other maladies, e.g., keratoconus. In addition, the insert may subtend an arcuate path in a third dimension around an axis which corresponds to the meridional radius. There is some interaction between the portions of the combination insert, but generally the thickness, length, and width of the sections may be varied to independently correct the noted vision acuity maladies.

FIG. 7A shows a front view of a cruciform-shaped variation (240) of the inventive insert having an inner radial leg (242) and a outer radial leg (244) as well as a circumferential portion (246). Again, the side view found in FIG. 7B shows an optional corneal radius of curvature such as discussed in relation to FIGS. 5A and 5B.

FIG. 8A shows a front view of a boomerang-shaped variation (250) of the inventive insert having a radial leg (252) and a circumferential portion (254). The side view found in FIG. 7B shows an optional radius of curvature. The radial leg (252) is not situated in such a way that it is placed in line with a meridian of the cornea but it can be conceptualized as being a distributed or functionally wider radial leg.

Further, the typical width of the individual inserts discussed above is often between 0.2 mm and 2.0 mm. The typical thickness is often between 0.15 mm and 0.5 mm. In addition to the width and thickness of the insert tapering at one or both ends, the thickness of the insert may optionally vary from one end to the other end of the insert (e.g., along the centroidal length of the insert) to provide for a desired change in corneal curvature at the location of the insert. The centroidal length of the insert (i.e., the length of the insert measured along the centroidal axis of the insert) is contemplated to rarely exceeds 3.0 mm. Preferably, the insert has a centroidal length which is less than or equal to 2.5 mm, and more preferably less than 2.0 mm. When the centroidal length is determined for an insert configuration other than the simple configuration shown in FIG. 4A (e.g., such as the insert shown in FIG. 4B), the centroidal length corresponds to the length of the radially arcuate portion measured along the centroidal axis of that portion. As another example (e.g., the insert of FIG. 4C), this length corresponds to the length of the generally radially extending leg (e.g., the non-circumferentially extending portion) measured along its centroidal axis. These parameters (along with certain other variables such as the cross-sectional shape of the device and its constituent polymers and stiffness) determine, in large part, the level of correction achievable by use of a selected insert.

Other non-limiting forms for the inventive insert are exemplified in FIGS. 9-15. FIG. 9 shows a perspective view of an insert (231) having a generally hexagonal cross section. The insert (239) is shown to be straight, that is, not to have a shape which conforms to the curvature of the anterior corneal surface prior to its introduction into the eye. Consequently, this variation would likely be produced of a material which is pliable and able to conform to a pocket previously formed in the cornea.

FIG. 10 shows a further variation (233) of the device in FIG. 9. This device (233) has a pre-form curve to it and, consequently, the inventive insert may be made of a stiff or pliable material.

FIG. 11 shows a square cross-section variation (235) of the device which is straight when not confined in the corneal channel. A gentle taper of the device (235) may be seen in the Figure. Either end of the device (25) and others described herein having tapered shapes, may be inserted into the eye using either the thin or fat end extending toward the outer periphery of the cornea. Typically, however, the fatter end will be placed at the outer periphery. The rate of taper from one end of the device is not important to this invention and need not be linear along the axis of the device, but may be of any convenient form.

FIG. 12 shows a further variation (237) of the device (235) shown in FIG. 11. In this instance, the device (237) is curved or has “preform” as well as having an axially variable cross-section.

The variations of this invention actually depicted in the drawings are considered to be only examples of the wide range of specific devices suitable for use in this inventive concept.

FIG. 13 shows a variation (239) of the invention similar in shape to the device shown in FIGS. 5A and 5B. This variation (239) and the others shown in FIGS. 14 and 15 have tapered ends to ease introduction of the device into the eye and lessen the trauma it may cause during that introduction and during use of the device. The insert (239) shown in FIG. 13 has generally smooth anterior and posterior surfaces and somewhat blunt opposing ends. It (239) is shown to be curved although such a form is not required.

FIG. 14 shows a variation of the devices shown in FIGS. 9 and 10 in which one or more of the ends (243) have a tapered or blunt shape.

Similarly, FIG. 15 shows a variation of the devices (245) shown in FIGS. 11 and 12 in which one or more of the ends have a tapered or blunt shape.

It should be apparent that these devices may be sterilized using known procedures having sterilants such as ethylene oxide or radiation (if the chosen materials so permit). The devices must be sterilized prior to use. It would be a normal practice to package these devices in ways using packages known for other ophthalmic devices capable of preserving the sterilization state. A typical commercial packaged, sterilized device would contain at least one device in such a sterile package. Depending upon the chosen materials for the insert, the packaging might be dry and include an inert gas or might contain a sterile fluid such as saline solution.

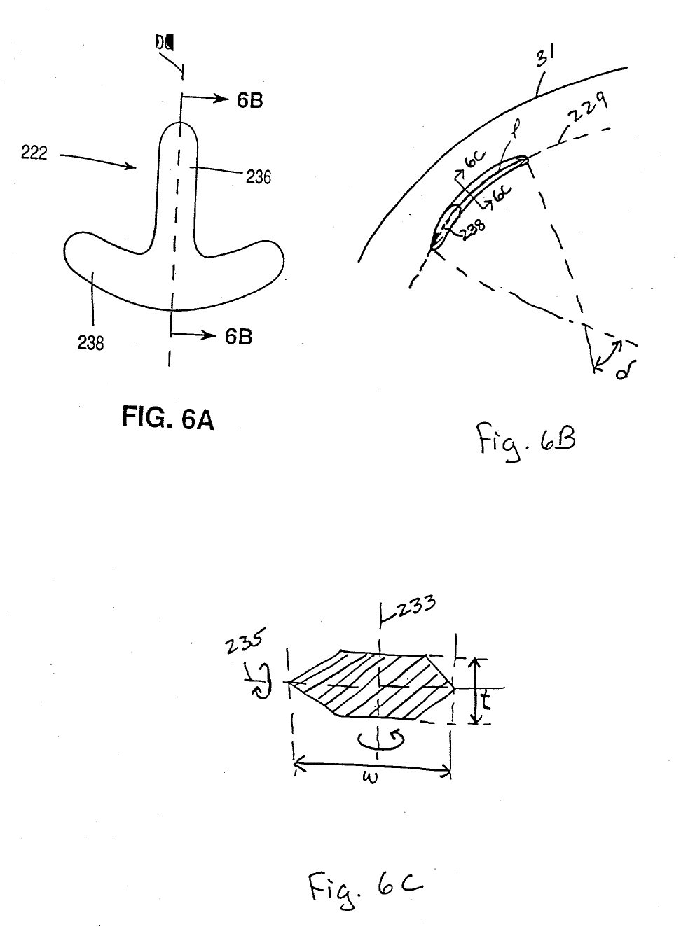

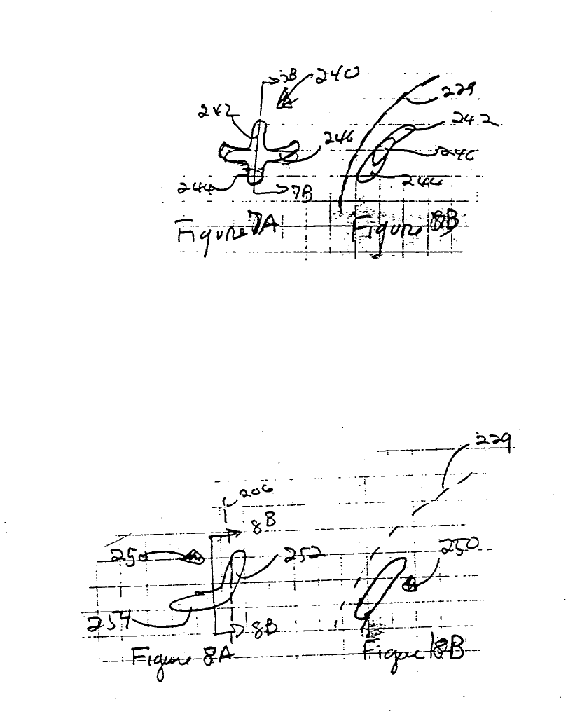

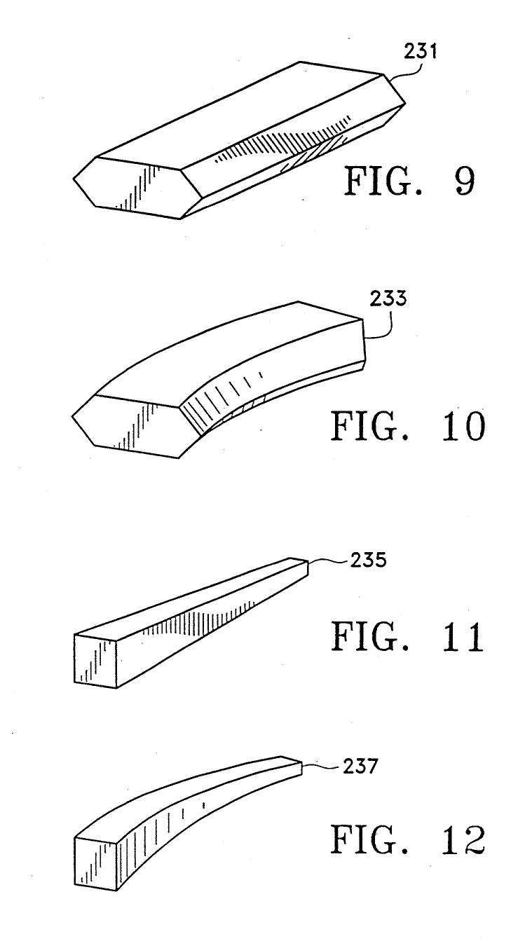

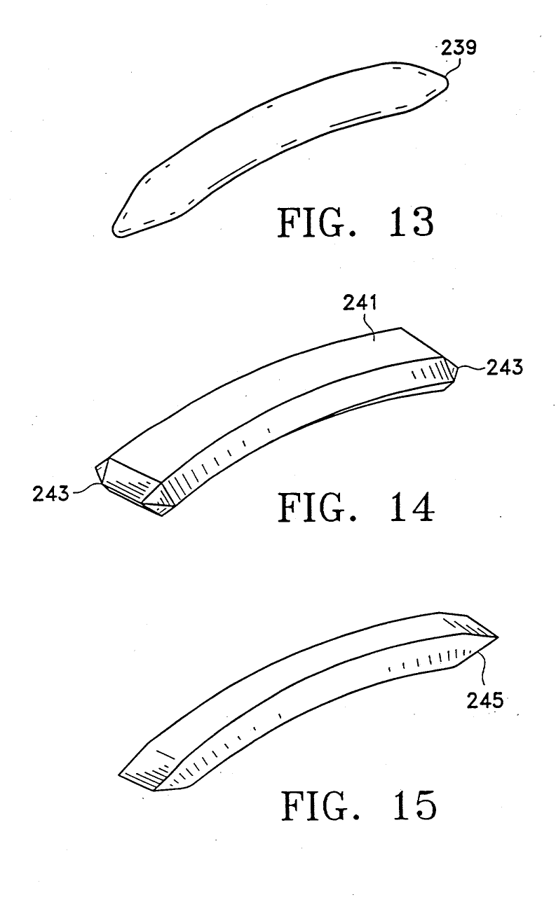

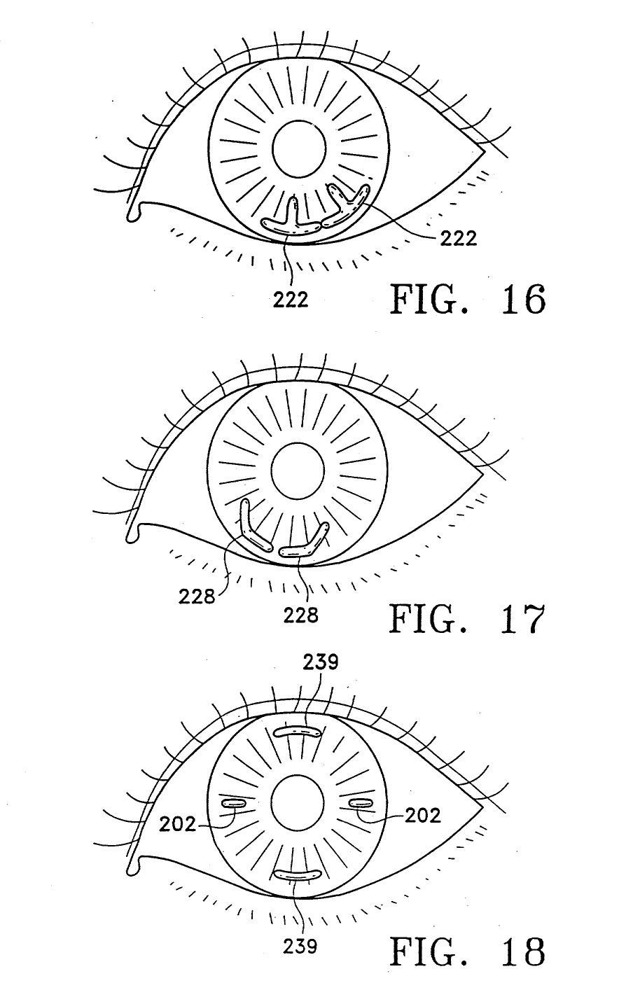

FIGS. 16 and 17 show variations of the invention in which multiple inserts are included in an intralamellar tunnel included within a human eye. FIG. 16 is intended to demonstrate a variation in which a number of inserts (222) are placed contiguously in an array within the channel rather than in an equally spaced array as was described in conjunction with FIG. 3A.

FIG. 17 shows a variation in which two inventive inserts (228) are nearly contiguous within the intrastromal channel.

FIG. 18 shows an array of two inventive radial inserts (202) in conjunction with two circumferentially placed intrastromal segments (239). The radial inserts (202) are placed in the cornea for the purposes noted above, typically hyperopia correction and perhaps astigmatism correction, and the circumferential segments (239) are introduced for myopia or astigmatism correction. A complete disclosure of the structure and use of the segments (239) may be found in U.S. patent application Ser. No. 08/101,438, entitled SEGMENTED PREFORMED INTRASTROMAL CORNEAL INSERT, filed Aug. 2, 1993 and Ser. No. 08/101,440, entitled SEGMENTED PLIABLE INTRASTROMAL CORNEAL INSERT, filed Aug. 2, 1993, both by Silvestrini, the entirety of which are incorporated by notice. The specific array of radial inserts (202) and circumferential segments (239) is not limited to the alternating pattern shown in the Figure, nor is the invention limited to the positioning or numbers of inserts shown in the Figure. The choice of and placement of appropriate insets and segments is left to the attending health professional based upon the abnormality to be treated.

The materials used in these inserts may be relatively stiff (high modulus of elasticity), physiologically acceptable polymers such as acrylic polymers like polymethylmethacrylate (PMMA) and others; polyfluorocarbons such as TEFLON; polycarbonates; polysulfones; epoxies; polyesters such as polyethyleneterephthalate (PET), KODAR, and Nylon; or polyolefins such as polyethylene, polypropylene, polybutylene, and their mixtures and interpolymers. Certain glasses are also suitable for the devices. By “high modulus of elasticity” is meant a modulus greater than about 3.5 kpsi. Many of these polymers are known in the art to be appropriately used in hard contact lenses. Obviously, any polymer which is physiologically suitable for introduction into the body is useful in the inserts of this invention. Many of the listed polymers are known to be suitable as hard contact lenses. For instance, PMMA has a long history in ophthalmological usage and consequently is quite desirable for use in these inserts.

Additionally, the polymeric material making up the insert may be one or more low modulus polymers, e.g., those having a modulus of elasticity below about 3.5 kpsi, more preferably between 1 psi and 1 kpsi, and most preferably between 1 psi and 500 psi, which are physiologically compatible with the eye. Most polymeric materials used in soft contact lenses are suitable the inserts of this invention. The class includes physiologically compatible elastomers and such polymers, typically crosslinked, as polyhydroxyethylmethylacrylate (Poly-HEMA) or polyvinylpyrrolidone (PVP), polyethylene oxide, or polyacrylates, polyacrylic acid and its derivatives, their copolymers and interpolymers, and the like as well as biologic polymers such as crosslinked dextran, crosslinked heparin, or hyaluronic acid. Acrylic polymers having a low T_{g} are also suitable.

In many instances, the intrastromal segments may be hybrid, that is to say, the segments are made up of a number of polymeric layers typically with a soft or hydratable polymer on their outer surface. These hybrid segments will be described with greater particularity below. Partially hydrated or fully hydrated hydrophilic polymers are typically slippery and consequently may contribute to the ease with which the insert may be introduced into the interlamellar tunnel. Suitable hydrophilic polymers include polyhydroxyethylmethacylate (PHEMA), N-substituted acrylamides, polyvinylpyrrolidone (PVP), polyacrylamide, polyglycerylmethacrylate, polyethyleneoxide, polyvinyl alcohol, polyacrylic acid, polymethacrylic acid, poly (N, N-dimethyl amino propyl-N¹-acrylamide) and their copolymers and their combinations with hydrophilic and hydrophobic comonomers, crosslinks, and other modifiers. Thermoplastic hydrogels include hydropolyacrylonitrile, polyvinyl alcohol derivatives, hydrophilic polyurethanes, styrene-PVP block copolymers and the like.

The intrastromal segment may be lubricated with suitable ocular lubricants such as hyaluronic acid, methylethyl cellulose, dextran solutions, glycerine solutions, polysaccharides, or oligosaccharides upon its introduction to help with the insertion particularly if one wishes to insert intrastromal segments of hydrophilic polymers without prior hydration. If a hybrid segment having a hydrophilic polymeric covering or a segment comprising a hydrophilic polymer is inserted into the eye without prior hydration, subsequent to the insertion, the intrastromal segment will swell to its final size or thickness within the eye. This swelling often permits the inclusion of larger intrastromal segments than would normally be accommodated within normal sized intrastromal channels.

Low modulus polymers used in this invention are often absorbent, particularly if they are hydratable, and may be infused with a drug or biologic agent which may be slowly released from the device after implantation of the intrastromal segment. For instance, the low modulus polymer may be loaded with a drug such as dexamethasone to reduce acute inflammatory response to implanting the device. This drug may help to prevent undesirable vascular ingrowth toward the intrastromal segment and improve the overall cosmetic effect of the eye with the insert and segment. Similarly, heparin, corticosteroids, antimitotics, antifibrotics, antiinflammatories, anti-scar-forming, anti-adhesion, and antiangiogenesis factors (such as nicotine adenine dinucleotide (NAD⁺)) may be included to reduce or prevent angiogenesis and inflammation.

Clearly, there are a variety of other drugs suitable for inclusion in the intrastromal segment. The choice will depend upon the use to which the drugs are placed.

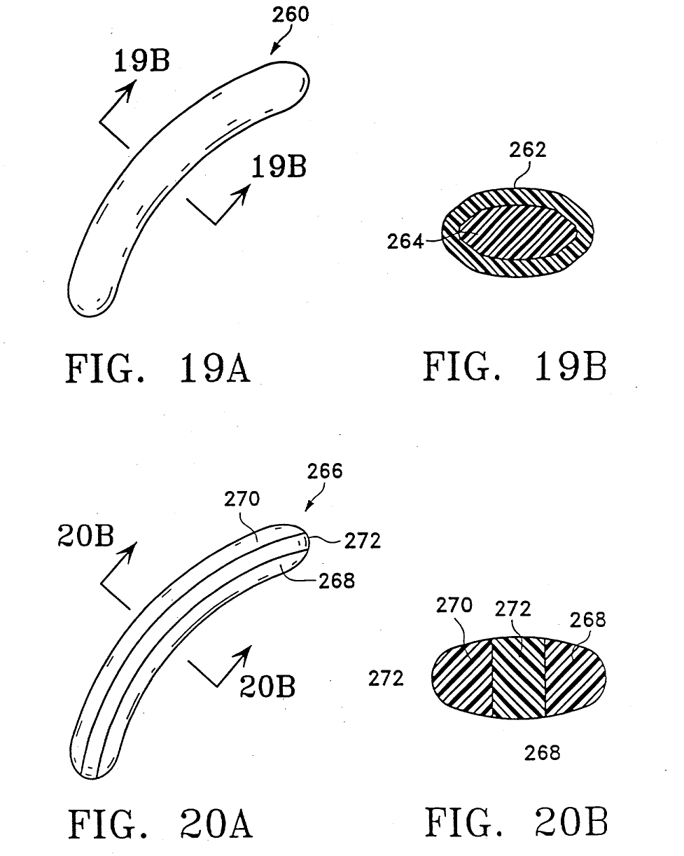

FIG. 19A is a side view of a variation of the intrastromal sector or insert (260) made of a low modulus polymer system or hydratable outer coating (262). FIG. 19B shows the inner cavity (264) of the insert. This intrastromal segment may be inserted into the intrastromal space created by the dissector as a covering on a tool similar to the dissector which created the intracorneal pocket or channel. Once in position the insertion tool is rotated out of the intrastromal segment leaving the shell within the stroma.

FIG. 19B shows the inner cavity (264) which may be filled with a biologic, a drug or other liquid, or biologically active eye treatment material. These devices may be tied or pinched or crimped or otherwise closed, typically at their point of insertion, by known techniques. If the inserts were closed or sealed prior to introduction, the insert may later be punctured with a syringe and a fluid or gel my be introduced or withdrawn for a variety of clinical reasons.

The shell (262) may be injected with a settable soft polymer core (264), allowed to expand to a desired thickness, and set. Polymeric gels which do not polymerize in situ are preferred. Suitable injectable polymers are well known but include polyHEMA hydrogel, cross-linked collagen, cross-linked hyaluronic acid, PVP, polyacrylonitriles, polyacrylamides, polyacrylic acids, their copolymers and terpolymers, vinyl alcohol derivatives, etc. Siloxane gels and organic-siloxane gels such as cross-linked methyl vinyl siloxane gels are acceptable but are generally considered to be less suitable.

The core (264) may also be a high or low modulus polymer if so desired.

FIG. 20A shows a front view of a hybrid layered intracorneal insert (266). Viewed in cross section in FIG. 20B, the multiple layers of the insert (266) may be seen. FIGS. 20A and 20B are intended to show the concept of a multilayered insert made up of polymers of different characteristics. In this example of a multi-layered insert, the hybrid insert has inner (268) and outer faces (270) of polymers having low moduli of elasticity.

The inner portion or core (272) may be a physiologically compatible polymer having a high modulus of elasticity or other polymer of low modulus.

If hydratable polymers are chosen for the outside layers, the extent to which those outer layers swell upon hydration is dependent upon the type of polymer chosen and, when the polymer is hydratable, upon the amount of cross-linking found in the outer layers (268) and (270), and upon the thickness of the layer. Generally speaking, the more highly linked the hydratable polymer, the smaller the amount of volume change upon hydration. Conversely, a polymer having only sufficient cross-linking for strength in the service in which this device is placed, will have a somewhat lower level of cross-linking. Alternatively, a substantially nonswellable polymer system may be formed of a hydrogel physically interpenetrated by another polymer which does not hydrate, e.g., PMMA into polyHEMA.

The thickness of the outer layer depends in large function upon the intended use of the intrastromal segment. If the outer layer is used to provide a swellable outer layer which does not add significantly to the size of the intrastromal segment or is used functionally as a lubricant layer, the other layer may be quite thin—even to the point of a layer of minimum coverage, perhaps as thin as a single molecular layer.

Of course, the inner and outer layers need not be, respectively, low modulus and high modulus polymers but may instead be multiple layers of low modulus polymers including an outer hydrophilic polymer layer and an inner hydrophobic polymer; a variety of hydrophilic polymers; etc.

Additionally, the inventive device shown in FIGS. 20A and 20B need not have a inner (268) and outer (270) layers over the entire insert. For instance, an insert having a thicker portion and a substantially thinner portion may be desired. An insert having an inner core of a high modulus polymer and an outer covering of a swellable polymer might be chosen. The surgeon would remove a portion of the insert's exterior coating or face prior to introducing the insert into the eye. Further, hydrophilic polymers are more easily infused with therapeutic and diagnostic materials than are the high modulus materials. In the variation just noted, the insert may then be used to deliver the infused therapeutic and diagnostic materials in a greatly delimited treatment or diagnostic area.

FIGS. 21 to 24 show procedures for introducing the inventive inserts into the cornea.

FIGS. 21A-21D show a procedure for introducing the inserts into the cornea using a circumferential intrastromal channel (300). The former portion of this general procedure is described in U.S. Pat. No. 5,300,118, to Silvestrini et al, issued Apr. 5, 1994, the entirety of which is incorporated by notice. In the first step illustrated in FIG. 21A, a small meridional incision (302) is made in the outer periphery of the anterior surface of the cornea. The slit may be circumferential if so desired. This slit (302) does not perforate the cornea but instead terminates within the cornea itself. See, for instance, the depth of placement for the inserts shown in FIGS. 3B and 3C. Next, a dissector, such as is shown in U.S. Pat. No. 5,403,335, to Loomas et al, issued Apr. 4, 1995, is then introduced into the initial incision (302) and rotated to form the circumferential interlamellar tunnel (300) as illustrated in FIG. 21B.

The next step, as illustrated in FIG. 21C, involves introducing a first radial insert through the initial slit (302), and into the intrastromal channel (300). The radial insert is then rotated into the desired meridional position within the channel (300), for example, at the 12:00 position as shown. Other inserts (306, 308, and 310) are introduced in the same fashion. The initial opening (302) is then closed by use of a suture, glue, staple, or by electrosurgical welding. The radial inserts may be introduced into the channel by way of the incision (302) in any appropriate manner. For example, the insert may be grasped and manipulated through the incision and into the channel using standard micro-forceps. Preferably, the forceps are constructed with tip ends having enhanced gripping features to positively hold the insert against sliding or rotatation relative to the tip ends. Such features may include recesses or indentations adapted to receive a portion of the insert, protuberences, gripping teeth, or other such features constructed to positively hold the insert. The exact configuration depends upon the shape of the insert and the preference of the surgeon.

Alternatively an introducer apparatus capable of holding and controllably inserting one or more inserts into the channel may be used. Suitable instruments for placing an insert into an intracorneal channel can be found in “CORNEAL IMPLANT INTRODUCER AND METHOD OF USE”, filed on Dec. 18, 1997 (Attorney docket no. 251692005200), the entirety of which is herein incorporated by reference.

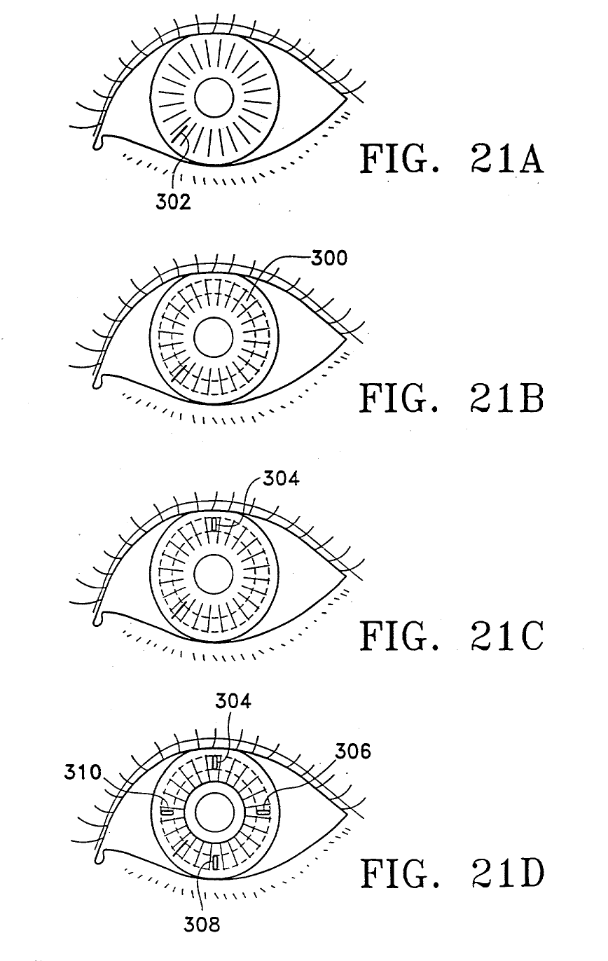

FIGS. 22A-22C show a procedure in which the various radial inserts are introduced using individual incisions. FIG. 22A shows the presence of four meridional incisions (312) made from the anterior surface of the cornea to a depth only partially through the cornea. Small pockets (314) are then meridionally placed from the four incisions (312) as shown in FIG. 22B. The four inserts (304, 306, 308, and 310) are then placed at the back end of the various pockets (314) as shown in FIG. 22C. Again, the various initial incisions (312) are then closed by use of sutures, glue, staples, or by electrosurgical welding.

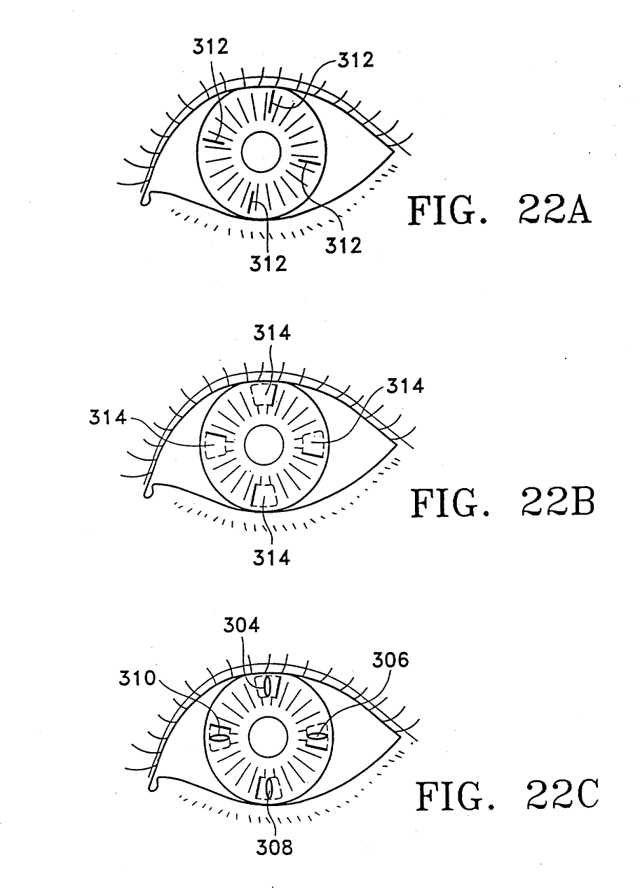

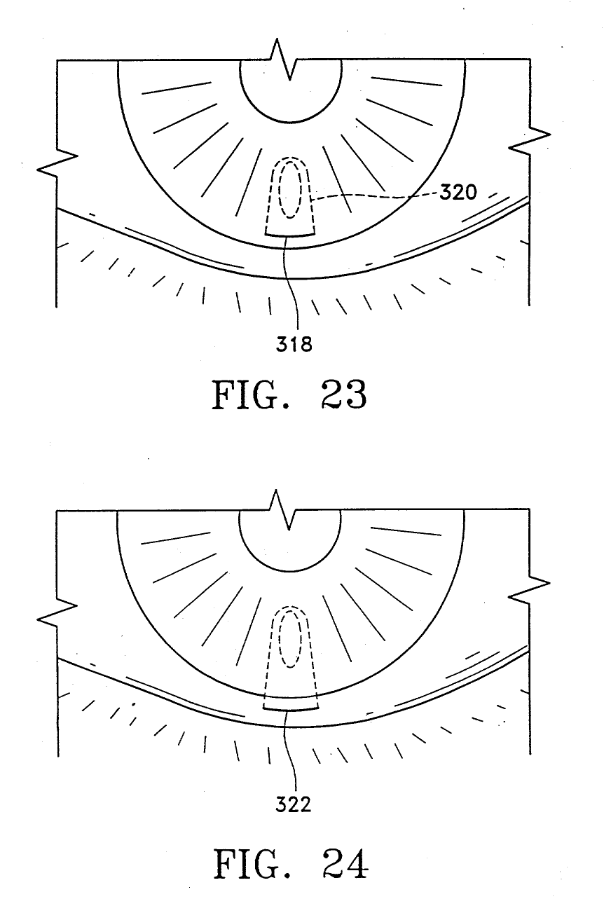

FIGS. 23 and 24 show entry incisions alternative to those shown in the procedure shown in FIG. 22. FIG. 23 shows a small circumferential incision (318) and a meridional pocket (320) for placement of the insert. Incision (318) is within the cornea. FIG. 24 shows an incision (322) outside of the cornea, within the limbus of the eye through which the insert is placed into the cornea.

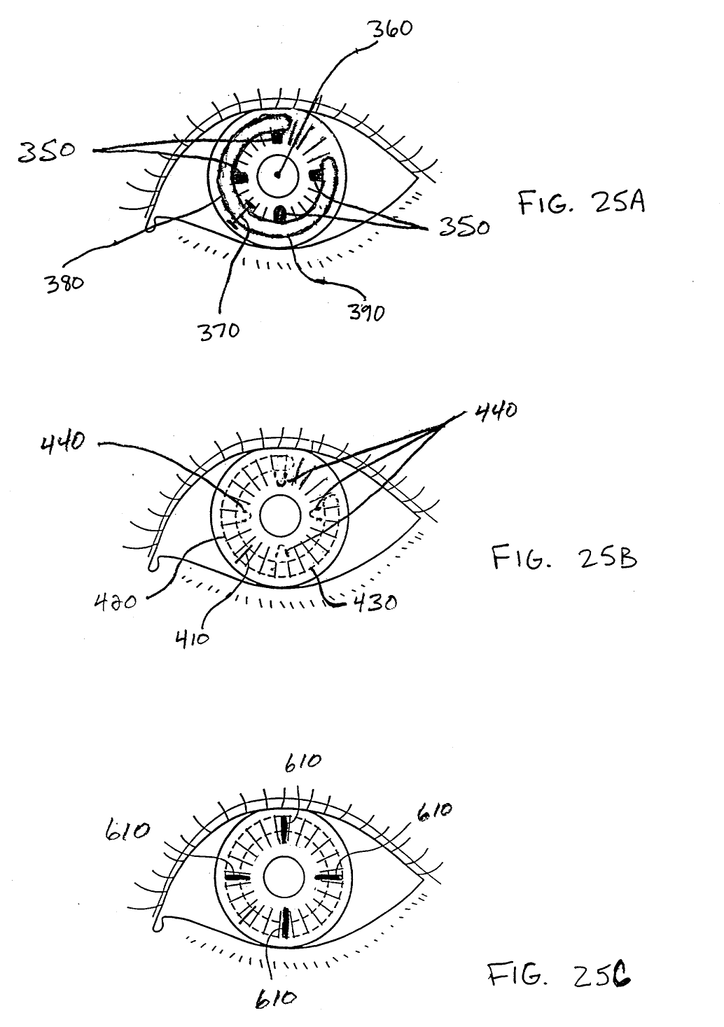

FIGS. 25A-25C illustrate a further exemplar procedure for introducing intracorneal inserts involving the combination of a circumferential channel and at least one radial pocket.

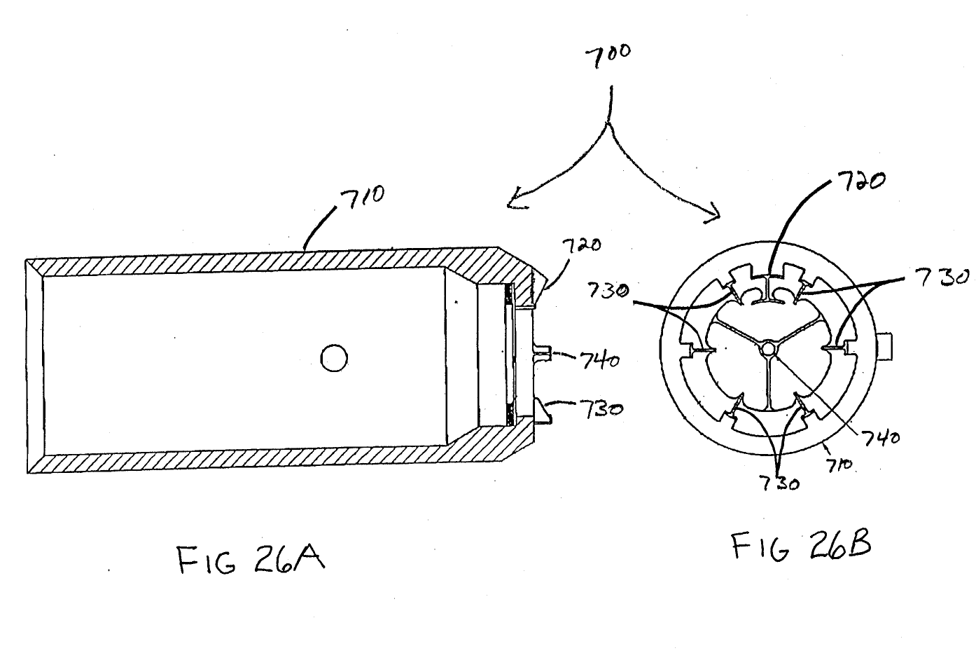

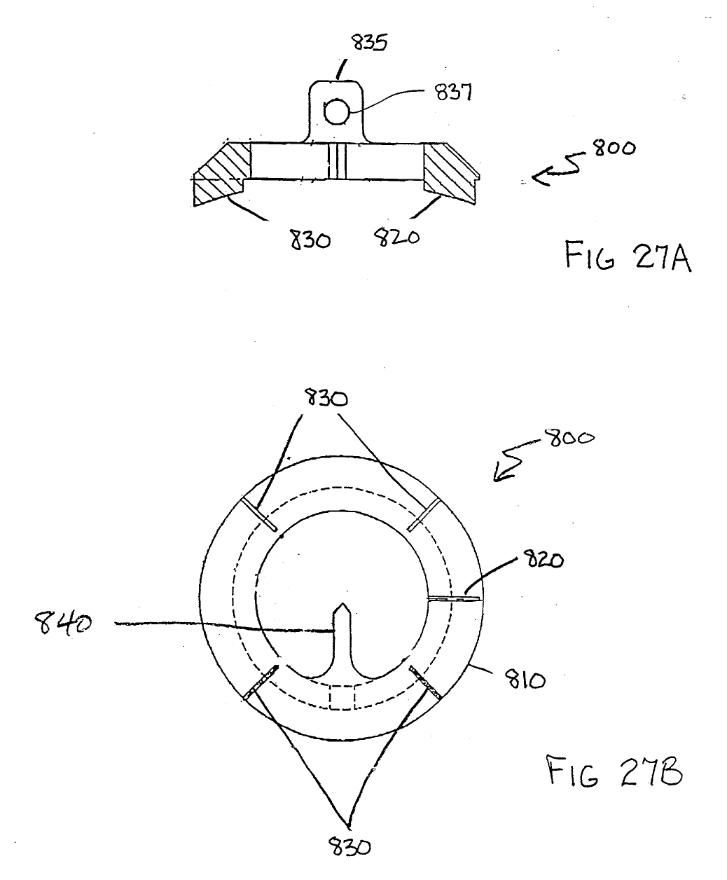

As illustrated in FIG. 25A, the surgeon places a center mark (360) at the geometric center of the cornea using a blunt instrument and an operating microscope or other comparable technique that accurately marks the center of the cornea. The surgeon next aligns a corneal marker (such as the one illustrated in FIGS. 26A and 26B or 27A and 27B, which is described in more detail infra) with the center mark and presses the corneal marker onto the cornea, marking the cornea with an incision mark (370), with clockwise (380) and counter-clockwise (390) circumferential channel marks, and with radial pocket marks (350).

The surgeon makes an incision (410 of FIG. 25B) into the cornea at the incision mark (370), cutting through some but not all of the stroma. The surgeon next forms clockwise (420), and counter-clockwise (430) circumferential channels between stroma.

Circumferential channels are formed using any of a number of methods. One method is disclosed in our U.S. Pat. No. 5,403,335, which is incorporated herein by reference in its entirety. In this method, a vacuum centering guide is positioned on the cornea using the centering mark on the cornea, and a vacuum of approximately 10-27 in. Hg is drawn to hold the vacuum centering guide on the eye. Small “starter” pockets are formed at the base of the incision perpendicular to the incision and in the direction that the circumferential channels are to be formed.

The incision is made using any appropriate surgical or diamond blade typically having a footplate on one or both sides of the blade to control the overall depth of the incision. Once the incision has been made, pocketing between corneal layers may be accomplished using a suitable instrument, such as a dissector or spreader as described in copending U.S. application Ser. No. 08/896,792 filed on Jul. 18, 1997 titled “OPTHALMOLOGICAL INSTRUMENTS AND METHODS OF USE” the entirety of which is herein incorporated by reference.

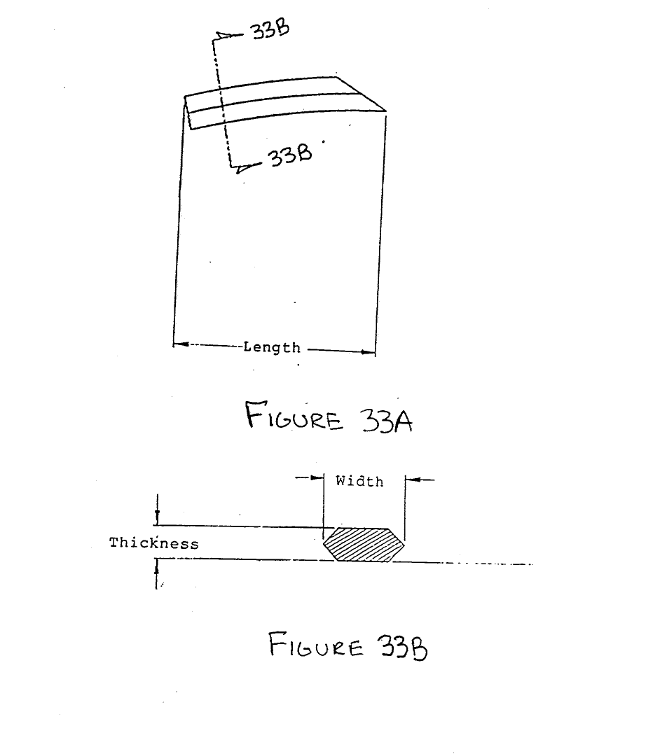

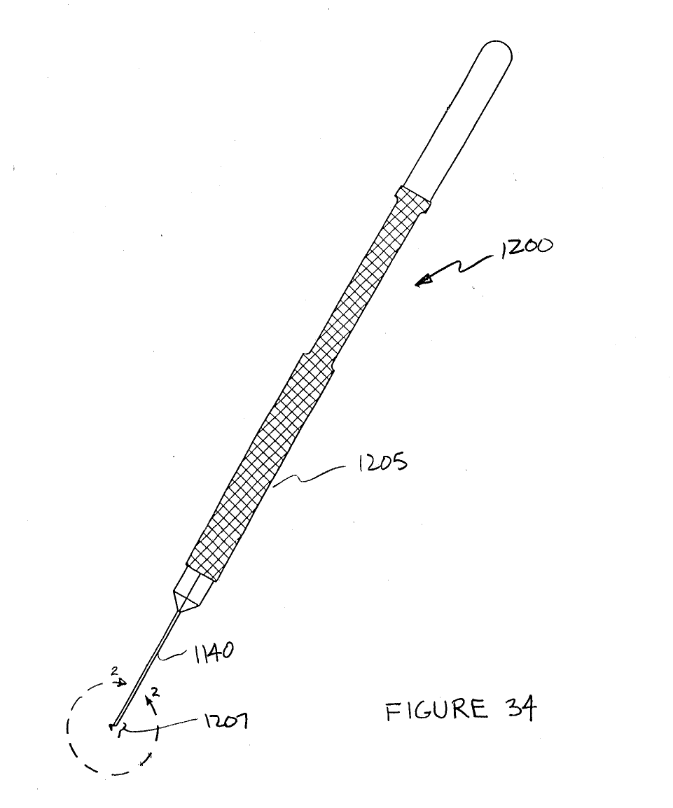

A specialized pocketing tool, such as those described in co-pending U.S. application titled “CORNEAL POCKETING TOOL”, filed on Dec. 18, 1997, the entirety of which is herein incorporated by reference, may also be used to separate the stromal layers at the appropriate depth at the base of the incision. Pocketing tool (1200), as illustrated in FIGS. 34-35, has an instrument handle (1205), a thin instrument shaft (1140), terminating distally in tip section (1207). Tip section (1207) is shown more clearly inserted into an incision (1610) in FIG. 35. Tip section (1207) or pocketing tool (1200) has a reference surface or region (1220) constructed to contact the surface of the cornea (1605). Reference region (1220), when in contact with the surface of the cornea (1605) ensures that the distal-most tip (1280) of the dissector or spreader section (1550) is adjacent to the base of the incision (1610).

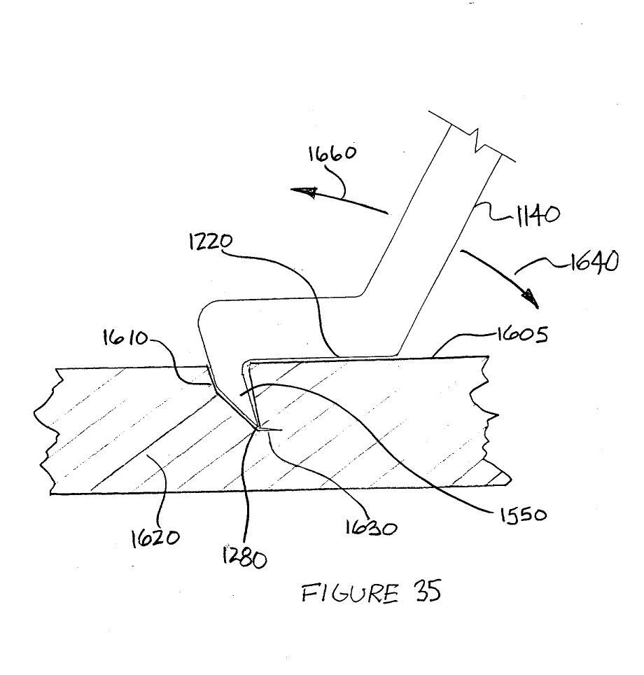

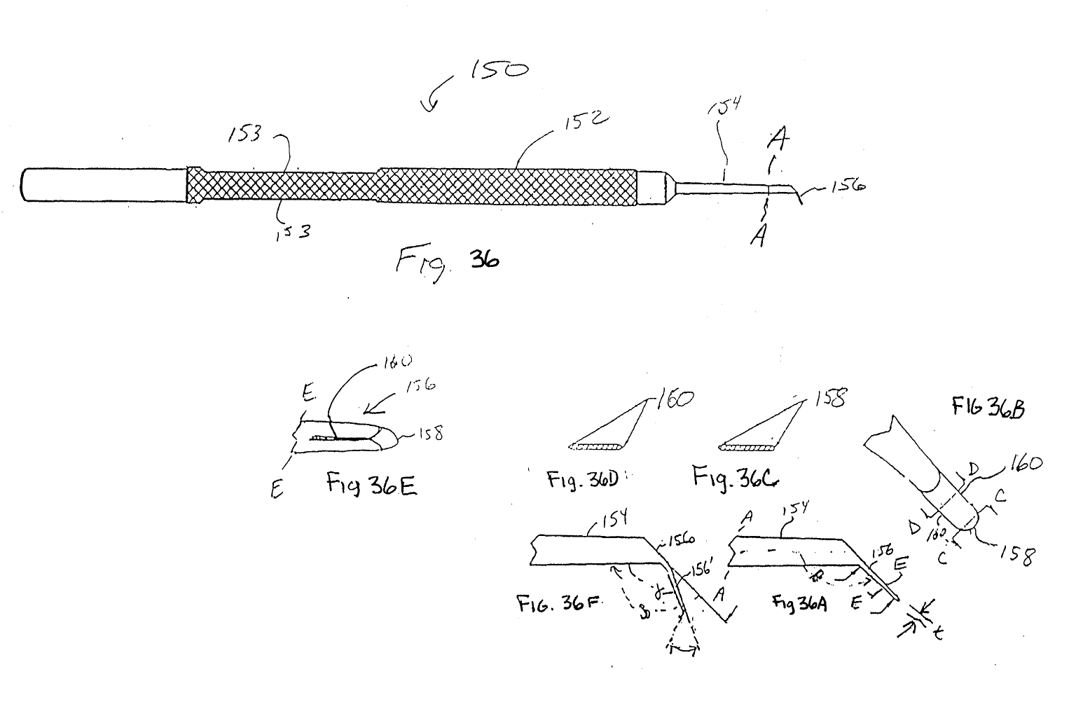

With the instrument in place as shown, the pocketing tool can be rotated in the direction of the arrow (1660) to create an intrastromal separation or pocket (1630). This small starter pocket may be enlarged as desired using a stromal spreader such as is described in co-pending U.S. application Ser. No. 08/896,792 filed on Jul. 18, 1997 titled “OPTHALMOLOGICAL INSTRUMENTS AND METHODS OF USE”, and described below with reference to FIGS. 36-38B.

Spreader 150 includes handle 152, extension 154, and tip 156. To provide increased rotational control of spreader 150, a portion of handle 152 is knurled and cutouts 153 are provided in opposing positions for marking the instrument. Extension 154 has a much smaller outside diameter than handle 152, and has a tapering outside diameter that gradually decreases toward the end of extension 154 that joins with tip 156.

Tip 156 is substantially flat and relatively wide and thin as observed in a comparison of FIGS. 36A and 36B. Tip 156 extends from extension 154 at an obtuse angle β to the longitudinal axis of extension 154 and handle 152, as shown in FIG. 36A. The obtuse angle provides the user with a comfortable handle position when tip 156 is inserted into the incision. Tip 156 has a tapering thickness t which decreases in the direction from the extension 154 to tip end 158.

As shown in FIG. 36B, tip end 158 is rounded and is preferably substantially hemispherical although greater and lesser radii of curvature may be employed to define the tip end. Importantly, the tip end is not knife sharp, but rather, is relatively blunt so as to function to separate tissue along layers, but not to cut. Tip end 158 transitions into tip sides 160 as the curvature of tip end 158 gradually straightens into the substantially straight edges of tip sides 160. Tip sides 160 are sharp, although not knife sharp. A comparison of the relatively dull edge of tip end 158 and the relatively sharp edges of tip sides 160 can be seen by comparing the sectional views of FIGS. 36C and 36D, respectively.

With the arrangement of stromal spreader tip 156 as described, the relatively dull, slightly rounded tip end 158 greatly reduces the risk of perforation of the corneal tissues upon insertion of the tip into the incision. Additionally, by rotating the spreader using handle 152 the stromal layers are can be effectively separated to form a pocket, or enlarge or otherwise modify an initial pocket created by the corneal pocketing tool described above.

FIG. 36E illustrates, in an exaggerated way, the transition between blunt tip end 158 and the relatively sharp edge of tip side 160, which supports the fact that the insertion of the tip presents a relatively low risk of perforation of the stromal tissues. Once the spreader has been inserted, separation can begin through use of sharper side edges 160, together with blunt tip end 158.

FIG. 36F shows a variation of the tip shown in FIG. 36A. In this variation, the joinder of tip 156 and extension 154 is formed at the obtuse angle β to the longitudinal axis of extension 154 and handle 152, the same as shown in FIG. 36A. However, the majority of the tip that is distal to the joinder of the tip and the extension, i.e., tip 156′ is formed at an angle γ with regard to the longitudinal axis of extension 154 and handle 152, and where angle γ is an obtuse angle that is less than obtuse angle β. The remaining features of tip 156′ are essentially the same as those described above with regard to tip 156 in FIGS. 36A-36E.

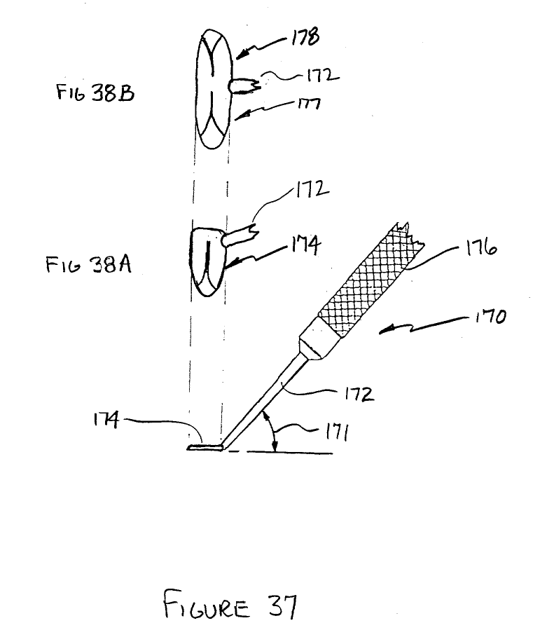

Preferably, the handle is oriented relative to the tip in such a way as to provide the surgeon with optimal visual and manual access to the surgical site. FIGS. 37-38B illustrate an alternative handle orientation. FIG. 37 illustrates a partial front view of spreader 170 having handle 176, extension 172 and spreader tip 174. Handle 176 may be at an angle 171 relative to the plane of spreader tip 174. Angle 171 is typically between about 20° to about 110°, more preferably between about 40° to about 70°, most preferably 60°.

FIGS. 38A and 38B show partial top views of spreader 170 illustrating a single spreader tip construction 174 and a double spreader tip construction 177,178 respectively. Because the single tip construction is asymmetrical, it may be desirable to have two opposite-handed instruments available for use depending on surgeon preference. The construction of FIG. 388B eliminates this need for two separate instruments. The spreader tips of FIGS. 37-38B may have any of the constructions described above.

Once the initial separation or pocket has been created in the manner described above, the surgeon inserts a clockwise dissector blade into the vacuum centering guide, and using a blunt-tipped instrument inserted into one of the small “starter” pockets, lifts the corneal tissue, and inserts the tip of the dissector blade into the starter pocket. The surgeon then rotates the dissector blade, which separates stroma and forms a clockwise circumferential channel between stroma. The surgeon removes the clockwise dissector blade and repeats the procedure using the counter-clockwise dissector blade to form a counter-clockwise circumferential channel. Separate, unjoined circumferential channels of any arc length or a continuous 360° channel can be formed using this method.

The surgeon forms a radial pocket (440) by inserting a radial pocket-forming instrument (such as the one illustrated in FIGS. 29A-29C, which is described in more detail infra) through the single incision and into the circumferential channel a sufficient distance that a tissue separator (such as a blade) on the instrument either is under one of the radial pocket marks (350) on the cornea that crosses the circumferential channel or is adjacent to one of the radial pocket marks that ends at or near the circumferential channel. The surgeon rotates the radial pocket-forming instrument or translates the instrument laterally so that the blade engages the sidewall of the circumferential channel and separates stroma to form a radial pocket connected to the circumferential channel and located beneath the radial pocket mark. The length, width, and shape of the pocket are determined by the size and shape of the blade. The surgeon rotates or translates the radial pocket-forming instrument in the opposite direction to remove the blade from the radial pocket and repositions the blade adjacent to another radial pocket mark to form another radial pocket. When all radial pockets have been formed, the radial pocket-forming instrument is withdrawn from the circumferential channel. The cornea is thus prepared to receive an intracorneal insert.

This method of preparing a cornea to receive an intracorneal insert as described above requires only one incision into the cornea. The remaining surgery to form the circumferential channel and the radial pocket and to implant the radial insert in the radial pocket is performed through the single incision into the cornea. Consequently, only one site through which foreign matter can gain entry to the eye must heal. Surgery proceeds rapidly, and suturing of the single incision is performed quickly. The likelihood of infection is reduced, and the likelihood of rapid healing of the epithelium is increased.

Once the surgeon has prepared the cornea to receive an intracorneal insert as described above, the surgeon places a radial insert, such as radial insert (216) of FIG. 4A into the circumferential channel through the incision in the cornea. The radial insert illustrated in FIG. 4A may have a width (220) about equal to the width of the radial pocket and a length (218) about equal to the sum of the length of the radial pocket and the width of the circumferential channel. Since this radial insert is longer than the width of the channel, the insert is best maneuvered within the circumferential channel by inserting the insert length-wise through the incision and into the circumferential channel and keeping the major axis of the insert approximately parallel to the sidewalls of the circumferential channel as the insert is manipulated within the circumferential channel.

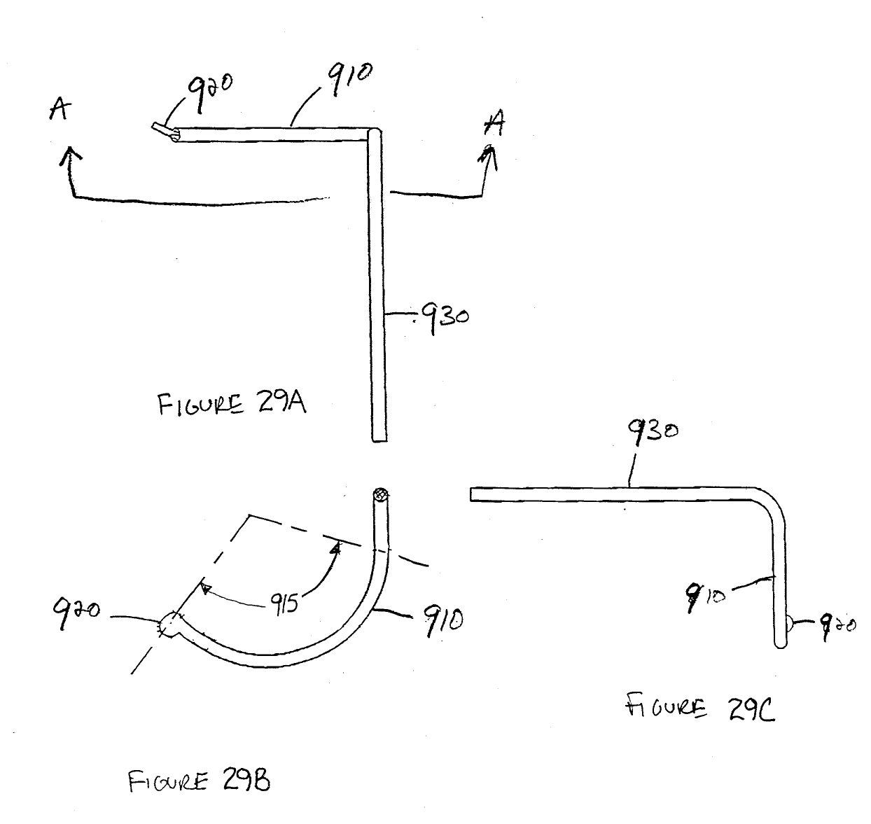

The surgeon may use an instrument to push or pull the radial insert to a position adjacent to a radial pocket, and then uses the same instrument or another positioning instrument (such as the one illustrated in FIGS. 30A and 30B, which is described in greater detail infra) to maneuver one end of the radial insert into the radial pocket and to maneuver the other end of the radial insert against the sidewall of the circumferential channel that is opposite to the radial pocket. If more than one radial pocket is connected to a circumferential channel, the surgeon places the first radial insert into the radial pocket that is located farthest from the single incision into the cornea. The surgeon places the next radial insert into the radial pocket that is second farthest from the single incision, and this process is repeated until the radial pockets have been filled with radial inserts (610), as shown in FIG. 25C.

The surgeon can insert short circumferential inserts between adjacent radial inserts, if desired. The short circumferential inserts allow the surgeon to further adjust the shape of the cornea and correct deficiencies in the patient's vision. In one method, the surgeon places a radial insert as discussed above into the farthest radial pocket from the single incision, and next the surgeon places a circumferential insert into the circumferential channel so that the circumferential insert abuts the radial insert. The circumferential insert is shorter than or the same length as the distance in the circumferential channel between adjacent radial pockets. The surgeon then alternately places a radial insert into the next farthest radial pocket and places a circumferential insert into the circumferential channel as described above until the surgeon has completed the surgical procedure. In another method, the surgeon inserts short radial inserts having lengths about equal to the lengths of the radial pockets into which the radial inserts are implanted. A single circumferential insert is placed into the circumferential channel to both hold the radial inserts in their pockets and to further reshape the patient's cornea. The number of inserts and the size and shape of each circumferential insert and radial insert are determined by the amount of reshaping of the cornea that is needed to provide a spherically-shaped cornea in the patient's eye.

Turning now to the specifics of the instruments discussed above, the corneal marker used to make the marks on the cornea to guide subsequent surgical procedures may be constructed in a number of ways. A corneal marker may be provided which has an incision marker, clockwise and counterclockwise channel markers, and radial pocket markers which form their corresponding marks simultaneously when the corneal marker is pressed against the patient's eye.

Alternatively, multiple corneal markers can be used to form the incision mark, the clockwise and counterclockwise circumferential channel marks, and the radial pocket marks which aid the surgeon during surgery. For example, two corneal markers and be used to form the desired marks. One corneal marker may have an incision marker, clockwise and counterclockwise channel markers, and a reticule or sight to enable the corneal marker to be aligned to the center mark (360) of the patient's cornea. The second marker may have radial pocket markers and a reticle or sight. Each corneal marker is individually aligned with the center mark (360) and pressed against the patient's cornea to form the desired marks. The combined incision/circumferential channel markers is usually pressed against the cornea before any vacuum centering guide is placed thereon so that the surgeon can easily make the initial incission into the cornea. After the vacuum centering guideis placed on the cornea, the surgeon inserts the second corneal marker into the vacuum guide and presses it against the patients cornea to from radial marks on the cornea to guide surgery.

A suitable corneal marker is illustrated in FIGS. 26A-26B. FIG. 26A is a side view and FIG. 26B is an end view of corneal marker (700). This corneal marker has a housing (710) to which incision markers, radial pocket markers, channel or pocket markers, and a positioner may be attached as desired. The incision marker and radial pocket markers are inked with a dye prior to aligning the marker to the center of the patient's cornea and pressing the marker to the patient's cornea to mark it with appropriate markings.

The positioner (740) is used to assure that any marks placed onto the cornea are placed at positions on the patient's cornea where the surgeon has specified that surgery will occur. Usually, the positioner is used to align the corneal marker with the center of the patient's cornea. This arrangement may be used in conjunction with a vacuum centering guide if desired. For the hand-held corneal marker (800) illustrated in FIGS. 27A and 27B, a reticle or sight (840) or other positioning means is used to position the corneal marker e.g. over a centering mark placed on the patient's cornea.

Radial pocket markers (730) mark the locations of radial pockets that will be formed within the patient's cornea. Radial pocket markers can be spaced equidistantly from adjacent radial pocket markers. Consequently, a four-pocket corneal marker has four radial pocket markers spaced 90° from one another; a five-pocket corneal marker has five radial pocket markers spaced 72° from one another, a six-pocket corneal marker has six radial pocket markers spaced 60° from one another, and so forth. Any number of radial pocket markers can be incorporated into the corneal marker, from one to ten or more.

The incision marker (720) is the “I” or “H” shaped marker of FIG. 26B which marks the site of the single radial incision to be made into the cornea from outside of the cornea. The incision marker can be located equidistant between adjacent radial pocket markers. For example, if the corneal marker has four equidistantly-spaced radial pocket markers, the incision marker is located 45° from its two adjacent radial pocket markers.

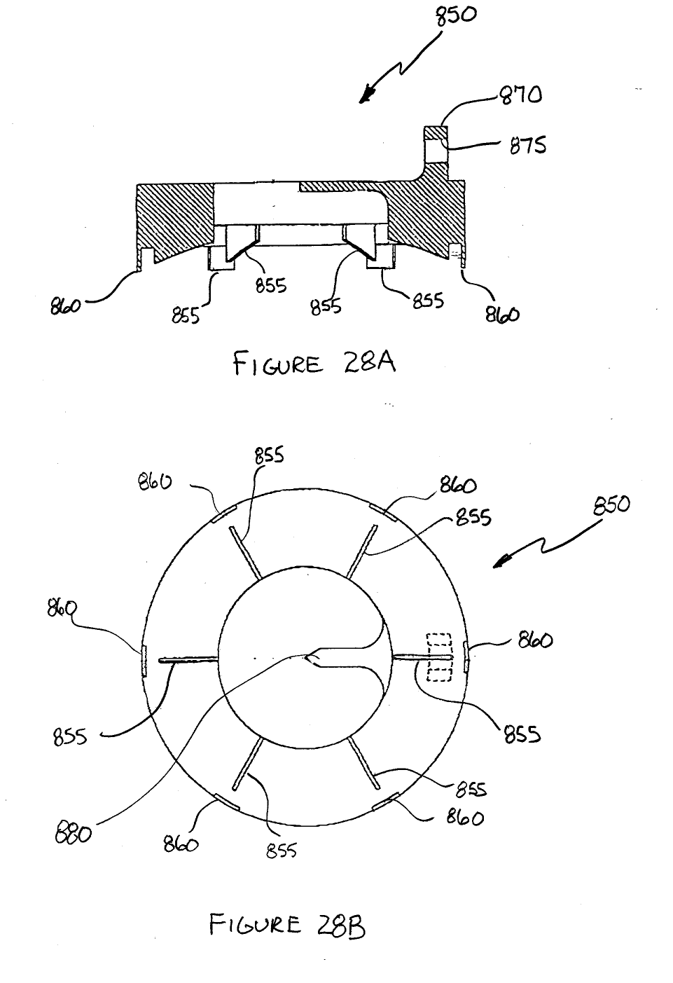

The corneal marker may have more than one incision marker, if desired. For example, if two unconnected circumferential channels are to be formed in the patient's cornea, the corneal marker will usually have at least two incision markers that provide the needed incision marks without having to align the corneal marker to the center of the patient's cornea and mark the cornea a second time. FIGS. 28A and 28B illustrate a hand-held corneal marker (850) having multiple circumferential incision markers (860) each corresponding to radial pocket markers (855). This type of marker is useful for the circumferential incision/radial pocket procedure illustrated in FIGS. 23 and 24.

The hand held markers (800, 850) are constructed to allow the attachement of a handle to allow easy manipulation by the surgeon. The instrument handle may have any comfortable shape and position that allows the surgeon to align the marker against the eye apply sufficient pressure to mark the cornea. The hand-held markers (800, 850) may be provided with handle mounting flanges (835 and 870, respectively) to which an instrument handle (not shown) may be attached using mounting holes (837 and 875, respectively).

The corneal marker can also have one or more circumferential channel markers which mark regions on the cornea where one or more circumferential channels will be formed. When the corneal marker has one or more circumferential channel markers, radial pocket markers can terminate on one side or the other of the circumferential channel marker, so that the radial pocket marks point generally toward the patient's pupil or point generally away from the patient's pupil. Or, the radial pocket markers can cross the circumferential channel markers to provide generally “X”- or cross-shaped marks. It is not necessary for the corneal marker to have a circumferential channel marker. For example, when clockwise and counter-clockwise dissector blades as described above and in U.S. Pat. No. 5,403,335 are used to form circumferential channels, the blades follow a predetermined path that is a function of the position of the vacuum centering guide over the cornea and the arc and position of the dissector blades on the dissecting tool. The length of the blades can establish the length of the circumferential channels, or alternatively the surgeon can watch the dissector blade and stop it at or slightly past the furthest radial mark that the dissector blade can reach. Marks from the circumferential marker are helpful in assuring that the dissector blades follow their intended path.

The radial pocket markers, incision markers, and circumferential channel markers are shaped to conform to the cornea. Consequently, these markers have curved faces that generally follow the curved shape of the cornea so that at least substantially all of the marking faces of these markers apply dye to the patient's cornea.

A corneal marker which has at least one incision marker, at least one radial pocket marker, and a positioner provides incision and radial pocket marks on the patient's cornea in the positions where surgery is to occur. The surgeon only needs to press the corneal marker to the patient's cornea once to mark the cornea with all of the marks the surgeon requires to perform the surgery described above. Surgical marks are correctly aligned to one another, which increases the reliability and accuracy of surgery.

The radial pocket-forming instrument as illustrated in FIGS. 29A-29C has a clockwise generally arcuate member (910), a tissue separator (920) on the generally arcuate member, and a handle (930) located at one end of the generally arcuate member. The radial pocket-forming instrument is inserted into a circumferential channel through the initial incision or incisions. The generally arcuate member follows the shape of the circumferential channel, so that the radial pocket-forming instrument can be inserted into the circumferential channel a distance that is sufficient to position the tissue separator at a site where a radial pocket is to be formed. The tissue separator is then pressed against a sidewall of the circumferential channel to separate stroma and form a radial pocket. The tissue separator is positioned generally on a radius through the center of the patient's pupil and the tissue separator faces away from (as illustrated in FIG. 29B) or toward the patient's pupil.

A circumferential channel typically has a radius of curvature of about or in excess of 3 mm at its edge closest to the pupil, and the circumferential channel typically has a radius of curvature of no more than about 4 mm on its edge furthest from the pupil. The generally arcuate member in this instance will have a radius of curvature of at least about 3 mm on its one side and less than about 4 mm on its other side, so that the generally arcuate member follows the shape of the circumferential channel.

Preferably, the radial pocket-forming instrument does not widen the circumferential channel as the instrument is positioned within the circumferential channel prior to forming a radial pocket. Consequently, the radial pocket-forming instrument of FIGS. 29A-29C has a width that is about equal to or is less than the width of the circumferential channel into which the instrument is inserted. The width of the instrument is the width of the generally arcuate member and the width of any tissue separator located at the site where the width of the generally arcuate member is measured. The width of the instrument is usually less than about 0.5 mm.

Clockwise and counter-clockwise radial pocket-forming instruments can be used to form the radial pockets when a single incision is used to form a circumferential channel or channels located on both sides of the single incision. A clockwise instrument has a generally arcuate member that travels in a clockwise direction from the handle to the tip of the instrument when viewing the generally arcuate member from directly above the handle of the instrument. A clockwise instrument can be inserted into a circumferential channel which was formed using a clockwise dissector blade.

The generally arcuate member of the radial pocket-forming instrument has an arc-length (915) measured from the center of the tissue separator. This arc-length must be sufficiently long that the tissue separator is able to reach the desired distance from the incision so that it can form a radial pocket at the desired site or sites around the channel. For example, in the instance where six equidistantly-spaced radial pockets are formed and a single incision is spaced equidistantly between two adjacent radial pockets, the arc-length of a generally arcuate member must be at least about 330° when the radial pocket-forming instrument has only one tissue separator. The arc-length does not have to be any more than about 150° when clockwise and counter-clockwise instruments are used to form radial pockets.

Alternatively, a number of radial pocket forming tools may be provided, each having an arc length only slightly longer than the distance to where a radial pocket is to be formed. For example, if inserts are to be placed every 60° from the initial incision, radial pocket forming tools having arc lengths in increments of 60° (about 30°, 90°, and 150°) would be provided. This advantageously prevents the surgeon from having to attempt to manipulate a pocket forming tool that has a large portion of its arc length outside of the incision.

In another aspect, the radial pocket-forming instrument can have more than one tissue separator on the generally arcuate member. The radial pocket-forming instrument can have, for example, as many tissue separators on the generally arcuate member as radial pockets that are to be formed in the portion of the circumferential channel in which the radial pocket-forming instrument will be inserted. The tissue separators will be located at positions on the generally arcuate member which correspond to the positions of the radial pockets when one of the tissue separators is aligned with the site where a radial pocket is to be formed. For example, in the instance where six equidistantly-spaced radial pockets are formed and a single incision is spaced equidistantly between two adjacent radial pockets, three tissue separators are located on e.g. a clockwise radial pocket-forming instrument at arc-lengths of about 30°, 90°, and 150°.

The tissue separator forms the radial pocket in or between stroma to allow the radial insert to be implanted therein. The tissue separator has a size and shape that are sufficient to form a radial pocket which holds at least a portion of the radial insert selected by the surgeon for implantation into that radial pocket. The tissue separator can be a blunt blade which separates stroma to allow insertion of the radial insert. Or, the tissue separator can be a sharp blade that cuts into the stroma. The tissue separator can be formed at an angle between e.g. about 20° and about 50° to the generally arcuate member to allow the tissue separator to better follow the curved contour of the stroma.

The tissue separator can form a radial pocket that has an angle intermediate between a radius drawn through the center of the cornea and a second line which is both tangential to the circumferential channel and perpendicular to the radius drawn through the center of the cornea. Thus, a radial pocket may not be located on a true radius from the center of the cornea but may, instead, be angled with regard to the true radius.

The positioning instrument fits within the circumferential channel and engages a radial insert to maneuver the insert into a radial pocket. The clockwise positioning instrument illustrated in FIGS. 30A and 30B has a generally arcuate member (1010), a tip (1020) positioned on the generally arcuate member, and a handle (1030) at one end of the generally arcuate member.

The size and shape of the generally arcuate member of the positioning instrument are very similar to the generally arcuate member of the radial pocket-forming instrument. The generally arcuate member of the positioning instrument has a width and shape which allow the generally arcuate member to be inserted into a circumferential channel without enlarging the channel significantly. Thus, the width of the member is about equal to or less than the width of the circumferential channel into which the generally arcuate member is to be placed, and in the embodiment illustrated in FIG. 10, the member is no more than about 0.5 mm wide. The generally arcuate member also has about the same radius of curvature as the circumferential channel, as described previously.

The tip (1020) on the positioning instrument is usually positioned at an end of the generally arcuate member. In FIGS. 30A and 30B, the tip is illustrated as a blunt end on a tapered wire forming the generally arcuate member, which wire was bent to an angle of about 90° to the plane of the generally arcuate member. The tip can be formed at any angle that allows the tip to maneuver the radial insert. The tip can be formed at an angle between 45° and 135°, for instance, and the tip can be bent upward or downward. The tip needs to be tall enough that the tip engages with a corner or side of a radial insert, so that the surgeon can coax or maneuver the radial insert into a radial pocket. The tip is preferably kept short so that the tip does not unduly drag against stroma as the positioning instrument is moved about in the circumferential channel. A tip height of 0.010-0.020 mm is sufficient to engage a radial insert to position it within a radial pocket. The tip may be smooth, or the tip may have small burrs or additional appendages such as arms which help to engage the radial insert when maneuvering it.

Clockwise and counter-clockwise positioning instruments can be supplied where a circumferential channel or channels extend on both sides of an incision into the cornea. The generally arcuate members of these instruments will typically have an arc length of 180° or less. As noted above, in the instance where six equidistantly-spaced radial pockets are formed and a single incision is spaced equidistantly between two adjacent radial pockets, a positioning instrument will have a generally arcuate member of an arc-length of no less than about 30°, and preferably the arc length is at least 90° or 150° so that the instrument can reach pockets that are distant from the incision

Of course, as noted above with regard to the pocket forming tool, a number of arcuate members may be provided, each having an arc length to extend a desired distance from the initial incision. Preferrably, the arc length of the arcuate member of the positioning instrument will be a little longer than the distance from the incision to the radial pocket of interest. For example, with radial pockets at 30°, 90° and 150°, arc lengths for the arcuate member of the positioning instrument may be 50°, 110°, and 170°. The added length is useful in case a segment is pushed beyond the radial pocket and it is necessary to hook on the far side of the insert to pull it back towards the incision.

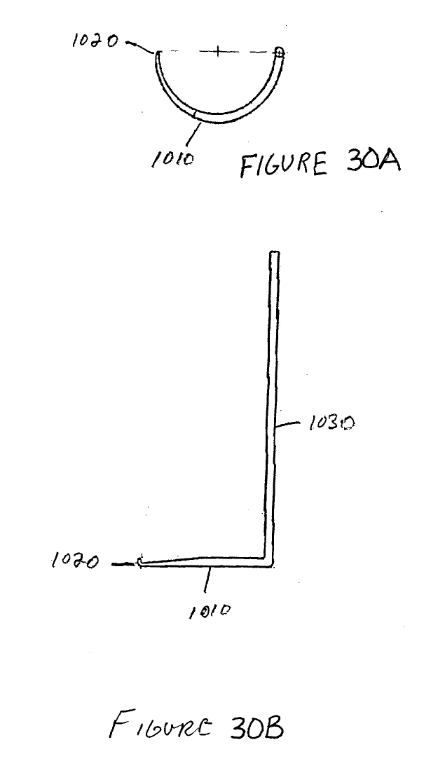

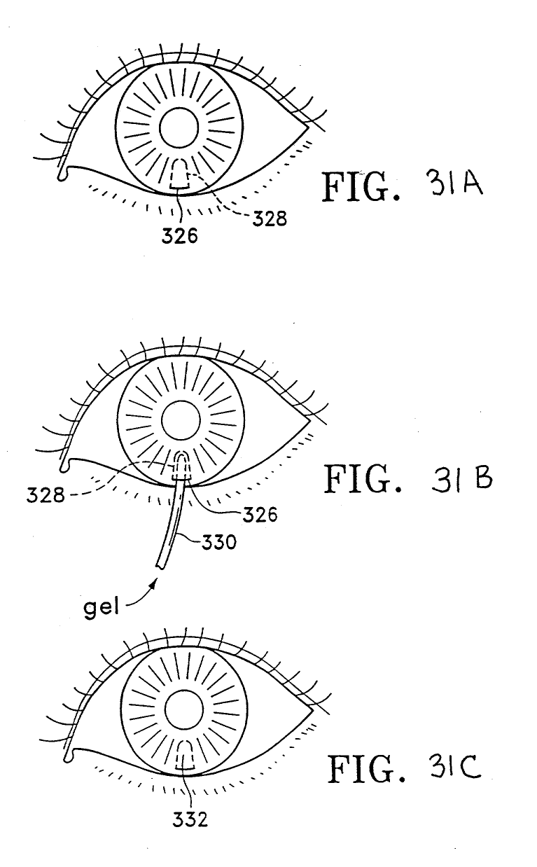

In addition to the devices which make up the invention and have been described above, this invention additionally includes the method of producing radial inserts comprising only a gel by using a method similar to the surgical procedures outlined above. FIGS. 31A-31C outlines such a procedure. In the first step, as illustrated in FIG. 31A, a small incision (326) is made into the cornea and a small meridional pocket (328) is formed. Next, a conduit (330) containing an appropriate gel is introduced into the incision (326) and the pocket (328) is filled with an appropriate amount of gel as shown in FIG. 31B. The gel radial insert (332) is shown in position in FIG. 31C. Incision (326) has obviously been closed.

### EXAMPLES

### Example 1

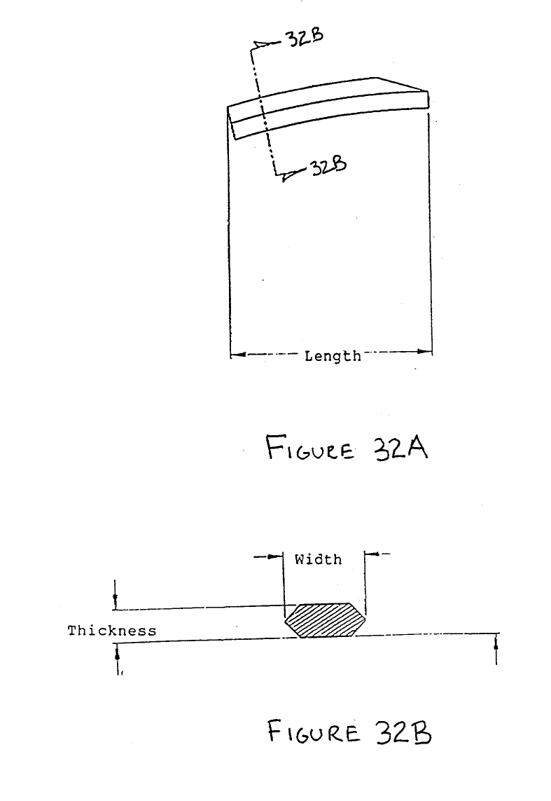

FIGS. 32A-32B and 33A-33B depict the shape of various inserts which were placed intracorneally into eyes from human cadavers. Two to six inserts were placed in each eye in the direction of a corneal meridian. The insert shown in FIGS. 32A-32B is partially tapered at one end in the direction of insertion, while the insert shown in FIGS. 33A-33B is fully tapered at one end. These inserts were preformed from polymethylmethacrylate. The dimensions of the inserts were as follows:

| | | Centroidal | | | Radius of |
|---|---|---|---|---|---|
| Insert | | Length | Width | Thickness | Curvature |
| No. | Insert Shape | (mm) | (mm) | (mm) | (mm) |
| Insert 1 | | 2.0 | 0.80 | 0.30 | 7 |
| Insert 2 | | 1.5 | 0.80 | 0.30 | 8 |
| Insert 3 | | 2.0 | 0.80 | 0.45 | 8 |
| Insert 4 | | 2.0 | 0.80 | 0.30 | 7 |
| Insert 5 | | 1.5 | 0.80 | 0.30 | 8 |
| Insert 6 | | 2.0 | 0.80 | 0.45 | 8 |

The length of the inserts were measured along their centroidal axes as they extended in the general direction depicted in FIGS. 32A and 33A, although the centroidal axes of the inserts are not shown.

Each of the inserts changed the corneal curvature by a desired amount up to 8 diopters to provide for hyperopic correction, and exhibited stable dimensions (no appreciable changes in length, width or thickness) over a time of one hour. Devices prepared in accordance with the foregoing example which were without curvature also changed the corneal curvature by approximately the same amount.

This invention has been described and specific examples of the invention have portrayed. The use of those specifics is not intended to limit the invention in any way. Additionally, to the extent that there are variations of the invention which are within the spirit of the disclosure and yet are equivalent to the inventions found in the claims, it is our intent that this patent cover those variations as well. All publications, patents and patent applications cited in this specification are incorporated herein by reference as if each such publication, patent or patent application were specifically and individually indicated to be incorporated herein by reference.

We claim as our invention:

1. An intracorneal insert for introduction into the cornea of a human eye, said insert comprising a physiologically compatible material and being adapted for implantation within a human cornea, said insert having a radius of curvature, measured along its centroidal axis, at least 5.0 mm.

2. The insert of claim 1 wherein said radius of curvature is at least 5.5 mm.

3. The insert of claim 1 wherein said radius of curvature is from 6.0 to 9.0 mm.

4. The insert of claim 1 wherein said radius of curvature is from 7.0 to 8.0 mm.

5. The insert of claim 1 wherein said radius of curvature approximates a human corneal curvature along a corneal meridian.

6. The insert of claim 1 where said insert comprises a low modulus physiologically compatible polymer.

7. The insert of claim 6 wherein the low modulus physiologically compatible polymer is selected from polyhydroxyethylmethylacrylate, polyvinylpyrrolidone, polyethylene oxide, or polyacrylates, polyacrylic acid and its derivatives, their copolymers and interpolymers, silicones, crosslinked dextran, crosslinked heparin, or crosslinked hyaluronic acid.

8. The insert of claim 7 wherein the low modulus physiologically compatible polymer is selected from polyhydroxyethylmethylacrylate and polyvinylpyrrolidone.

9. The insert of claim 6 wherein the low modulus physiologically compatible polymer is selected from hydratable polymers which swell upon hydration, hydratable polymer systems which do not swell upon hydration, and elastomers.

10. The insert of claim 6 wherein the low modulus, physiologically compatible polymer comprises an elastomer.

11. The insert of claim 1 where said insert comprises a polymer having a high modulus of elasticity.

12. The insert of claim 11 wherein the polymer is selected from polymethylmethacrylate; fluorocarbon resins; polysulfones; polycarbonate; epoxies; and polyolefins selected from polyethylene, polypropylene and polybutylene.

13. The insert of claim 12 wherein the polymer comprises polymethylmethacrylate.

14. The insert of claim 1 having a hollow inner portion.

15. The insert of claim 14 wherein the hollow inner portion is filled with a liquid.

16. The insert of claim 15 wherein the hollow inner portion is at least partially filled with a gel or a settable polymer.

17. The insert of claim 16 wherein the gel or settable polymer is selected from polyhydroxyethylmethacrylate hydrogel, cross-linked collagen, cross-linked hyaluronic acid, siloxane gels, polyvinyl pyrrolidone, and organic-siloxane gels.

18. The insert of claim 17 wherein the gel or settable polymer is polyvinyl pyrrolidone.

19. The insert of claim 14 wherein the hollow portion is at least partially filled with a drug or biologic agent.

20. The insert of claim 19 wherein the drug is selected from dexamethasone, heparin, corticosteroids, antimitotics, antifibrotics, antiinflammatory, anti-scar-forming, anti-adhesion, antithrombogenic, and antiangiogenesis factors.

21. The insert of claim 20 wherein the drug is an anti-inflammatory or antithrombogenic.

22. The insert of claim 6 additionally comprising a drug or biologic agent.

23. The insert of claim 22 wherein the drug is selected from dexamethasone, heparin, corticosteroids, antimitotic, antifibrotics, antiinflammatories, anti-scar-forming, anti-adhesion, antithrombogenic, and antiangiogenesis factors.

24. The insert of claim 2 comprising an anti-inflammatory or antithrombogenic.

25. The insert of claim 1 additionally comprising an ocular lubricant.

26. The insert of claim 25 wherein the ocular lubricant is selected from hyaluronic acid, methylethylcellulose, dextran solutions, glycerine solutions, polysaccharides, or oligosaccharides.

27. The insert of claim 1 comprising at least two polymeric layers.

28. The insert of claim 27 wherein at least one polymeric layer comprises a low modulus physiologically compatible polymer.

29. The insert of claim 27 wherein at least one polymeric layer comprises a high modulus physiologically compatible polymer.

30. The insert of claim 1 wherein the insert includes a portion having a length to width ratio of at least 1:1.

31. The insert of claim 1 having a predetermined shape.

32. The insert of claim 1 being constructed to substantially retain its shape over time after implantation within the cornea.

33. The insert of claim 1 being configured and adapted to alter the topography of the cornea in a manner that effects correction of a predetermined refractive disorder of the eye.

34. The insert of claim 1 being configured and adapted to alter the shape of the cornea by a predetermined amount.

35. The insert of claim 1 wherein said insert is radially arcuate.

36. An intracorneal insert for introduction into the cornea of a human eye, said insert comprising a physiologically compatible material and being adapted for implantation within a human cornea, said insert being without curvature along its centroidal axis

37. The insert of claim 36 being configured and adapted to alter the topography of the cornea in a manner that effects correction of a predetermined refractive disorder of the eye.

38. The insert of claim 36 being configured and adapted to alter the shape of the cornea by a predetermined amount.

39. The insert of claim 36 having a predetermined shape.

40. The insert of claim 36 being constructed to substantially retain its shape.

41. An intracorneal insert for introduction into the cornea of a human eye, said insert comprising a physiologically compatible material and being configured and adapted for implantation in a human cornea, said insert having a length measured along its centroidal axis of less than or equal to 2.5 mm.

42. The insert of claim 41 being configured and adapted to alter the topography of the cornea in a manner that effects correction of a predetermined refractive disorder of the eye.

43. The insert of claim 41 being configured and adapted to alter the shape of the cornea by a predetermined amount.

44. The insert of claim 41 having a predetermined shape.

45. The insert of claim 41 being constructed to substantially retain its shape.

46. The insert of claim 41 wherein said insert has a radius of curvature, measured along its centroidal axis, of at least 5.0 mm.

47. The insert of claim 46 wherein said radius of curvature is from 6.0 to 9.0 mm.

48. The insert of claim 46 wherein said his of curvature is from 7.0 to 8.0 mm.

49. The insert of claim 41 wherein said length is less than or equal to 2.0 mm.

50. A procedure for introducing an intrastromal insert into a cornea of a mammalian eye comprising the steps of:
a) making an initial incision in or near the cornea; and
b) introducing a biocompatible radially arcuate insert comprising a physiologically compatible polymeric segment through said initial incision in a direction along a meridian of the cornea.

51. The procedure of claim 50 wherein the initial incision is along a circumference of the cornea.

52. The procedure of claim 50 wherein the initial incision is along a meridian of the cornea.

53. The procedure of claim 50 wherein the initial incision is in the sclera of the eye.

54. The procedure of claim 50 comprising the additional step of producing an intrastromal intracorneal channel in the cornea from the initial incision prior to introducing the biocompatible radially arcuate insert into said initial incision.

55. The procedure of claim 50 comprising the additional step of producing at least one additional incision in said cornea for introducing at least one additional biocompatible radially arcuate insert into said cornea.

56. The procedure of claim 50 comprising the additional step of introducing at least one additional radial insert into said at least one additional incision.

57. A procedure for introducing a biocompatible gel into a cornea of a mammalian eye comprising the steps of:
a) making an initial incision in or near the cornea; and
b) introducing a biocompatible gel through said initial incision in a direction along a meridian of the cornea.

58. The procedure of claim 45 wherein the initial incision is along a circumference of the cornea.

59. The procedure of claim 45 wherein the initial incision is along a meridian of the cornea.

60. The procedure of claim 45 wherein the initial incision is in the sclera of the eye.

61. The procedure of claim 57 comprising the additional step of producing an intrastromal intracorneal channel in the cornea from the initial incision prior to introducing the biocompatible radially arcuate insert into said initial incision.

62. The procedure of claim 57 comprising the additional step of producing at least one additional incision in said cornea for introducing at least one additional biocompatible radially arcuate insert into said cornea.

63. The procedure of claim 57 wherein the gel is selected from the group consisting of hydrogel, cross-linked collagen, cross-linked hyaluronic acid, polyvinylpyrrolidone, polyacrylonitriles, polyacrylamides and polyacrylic acids.

64. The procedure of claim 57 comprising the additional step of forming a pocket in a direction along a meridian of the cornea from said incision.

65. An intracorneal insert for introduction into the cornea of a human eye, said insert comprising a physiologically compatible material and being adapted for implantation within a human cornea, said insert having a first elongated portion and a second elongated portion extending therefrom.

66. The insert of claim 65 wherein said first elongated portion has a second and a third elongated portion extending therefrom.

67. An intracorneal insert for introduction into the cornea of a human eye, said insert comprising a physiologically compatible material and being adapted for implantation within a human cornea, said insert having a boomerang shape.
